(19) United States
(12) Patent Application Publication
(10) Pub. No.: US 2024/0075185 A1
(43) Pub. Date: Mar. 7, 2024

(54) Extracellular matrix hydrogel derived from decellularized nucleus pulposus to alleviate orthopedic pain

(71) Applicant: The Board of Regents of the University of Nebraska, Lincoln, NE (US)

(72) Inventors: Rebecca Wachs, Lincoln, NE (US); David Lillyman, Lincoln, NE (US); Logan Piening, Lincoln, NE (US); Fei San Lee, Lincoln, NE (US)

(21) Appl. No.: 18/445,466

(22) Filed: Aug. 31, 2023

Related U.S. Application Data
(60) Provisional application No. 63/374,173, filed on Aug. 31, 2022.
(60) Provisional application No. 63/374,186, filed on Aug. 31, 2022.

(51) Int. Cl. A61L 27/36; A61L 27/24; A61L 27/52

(52) U.S. Cl. CPC A61L 27/3612; A61L 27/24; A61L 27/3654; A61L 27/3658; A61L 27/3687; A61L 27/52; A61L 2400/06; A61L 2430/24; A61L 2430/40

(57) ABSTRACT

Provided are compositions and methods of making a hydrogel-forming composition comprising decellularized porcine nucleus pulposus tissue that is solubilized and supplemented with collagen, then neutralized and thermally gelled in the presence of genipin. The hydrogel-forming composition may be injected into a joint or surrounding tissue of a subject having pain or inflammation. The pain or inflammation may be caused by degeneration of the intervertebral disc (IVD), wherein administration of the hydrogel-forming composition into the IVD may restore the lost disc volume and alleviate the pain and inflammation.

### CROSS-REFERENCE TO RELATED APPLICATIONS

This application claims priority to U.S. Provisional Patent Application No. 63/374,173, filed Aug. 31, 2022, and entitled “Neuroinhibitory Porcine Nucleus Pulposus Tissue for Disc Tissue Engineering”, and U.S. Provisional Patent Application No. 63/374,186, filed Aug. 31, 2022, and entitled “Extracellular Matrix Hydrogel Derived from Porcine Nucleus Pulposus to Alleviate Low Back Pain”, the entire contents of which are incorporated herein by reference.

### TECHNICAL FIELD

This disclosure relates to compositions and methods related to hydrogel-forming compositions having decellularized nucleus pulposus tissue (dNP), collagen, and an optional crosslinking agent, and more particularly, to hydrogel-forming compositions used for reducing, preventing, or treating pain or inflammation in a subject.

### BACKGROUND OF THE INVENTION

Chronic pain is a global socioeconomic crisis compounded by an absence of reliable, curative treatments. Chronic pain is a pervasive medical condition affecting millions of individuals worldwide. Various underlying causes, including neurological disorders, musculoskeletal conditions, and inflammatory processes, contribute to the development of chronic pain. This condition not only compromises the quality of life of those afflicted but also places a substantial economic burden on healthcare systems.

While many forms of chronic pain exist, one of the leading causes of chronic pain has been associated with pain stemming from the low back. The predominant pathology associated with chronic low back pain is degeneration of intervertebral discs in the lumbar spine. During degeneration, nerves can sprout into the intervertebral disc tissue and be chronically subjected to inflammatory and mechanical stimuli, resulting in pain. Pain arising from the intervertebral disc, or disc-associated pain, is a complex, multi-faceted disorder.

A study performed in 2013 indicates patients who develop chronic low back pain display remarkable increases in self-reported anxiety, depression, and stress. Furthermore, those suffering chronic low back pain report harmful effects on their exercise, personal life, social life, work relationships, and family relationships. Many studies have also reported tangential findings including decreased sleep quality and increased suicide in patients with chronic low back pain. Altogether, chronic pain imposes costs on society that must be remedied. Part of the difficulty in treating patients with chronic low back pain is that the pain can arise from multiple sources in the low back. This multifactorial nature of chronic low back pain has led researchers to further explore low back skeletal structures in hopes of finding reoccurring sources of pain.

For patients diagnosed with disc-associated pain and those suffering chronic low back pain suspected to be disc-associated, four different treatment modalities can be used to alleviate suffering. These four interventions include non-steroidal anti-inflammatory drugs (NSAIDs), non-pharmacological treatments, opioids, and surgery, however, each treatment has limitations. Unfortunately, NSAIDs are palliative fixes for pain and may result in gastrointestinal toxicity when chronically administered. Due to this limitation, NSAIDs are recommended only for short treatment and at the lowest dose possible. With regard to non-pharmacological treatments, systematic reviews have indicated that only highly managed, combinatorial non-pharmacological treatments, such as biopsychosocial rehabilitation, can have any effect on the management of low back pain. Further, while opioids have powerful analgesic capacity, they are typically avoided due to the high potential for abuse. Surgical fusion may be a last-line option for individuals suffering disc-associated pain, however, surgeries are highly invasive and ramifications may include fusion failure, increased chronic pain, adjacent spinal segment degeneration, and infection. Therefore, current non-invasive treatments provide inadequate long-term applicability, and current invasive treatments predispose patients to a myriad of surgical complications with inconsistent pain alleviation. Therefore, there remains a need for therapeutics that can specifically target underlying causes of pain, modulate pain signaling pathways, and promote long-lasting relief without significant side effects.

### SUMMARY OF THE INVENTION

Provided herein are compositions and methods of making a hydrogel-forming composition. The hydrogel-forming compositions comprise a decellularized nucleus pulposus tissue (dNP) and collagen. In some embodiments, the composition may further comprise a crosslinking agent. Beneficially, the hydrogel-forming compositions may be used for treating pain or inflammation in a subject, including, but not limited to the prevention, elimination, or reduction of symptoms of pain or inflammation in the subject.

In Example 1, a hydrogel-forming composition comprises a decellularized nucleus pulposus tissue (dNP), and collagen, wherein the composition is a hydrogel-forming composition.

Example 2 relates to the composition according to Example 1, wherein the dNP is present in the composition in an amount of between about 1 mg/mL to about 15 mg/mL.

Example 3 relates to the composition according to Example 1, wherein the collagen comprises type I collagen, and is present in the composition in an amount of from about 1 mg/mL to about 9 mg/mL.

Example 4 relates to the composition according to Example 1, wherein the composition further comprises a crosslinking agent comprising a genipin crosslinking agent, a lysyl oxidase (LOX) crosslinking agent, or a combination thereof.

Example 5 relates to the composition according to Example 4, wherein when the crosslinking agent comprises the genipin crosslinking agent, the genipin crosslinking agent is present in the composition in an amount of from about 0.1 mM to about 20 mM, and wherein when the crosslinking agent comprises the LOX crosslinking agent, the LOX crosslinking agent is present in the composition in an amount of from about 0.02 ng/mL to about 2 ng/mL.

Example 6 relates to the composition according to Example 1, wherein the dNP is a digested dNP, wherein the digested dNP is formed via a treatment of the dNP with an enzyme comprising pepsin in a concentration range of between about 1 mg/mL to about 10 mg/mL.

Example 7 relates to the composition according to Example 1, wherein the composition has a pH of between about 6 and about 8.

In Example 8, a method of treating pain or inflammation in a subject comprises administering a hydrogel-forming composition to a subject, wherein the composition comprises decellularized nucleus pulposus tissue (dNP) and collagen, wherein the administration of the composition to the subject prevents, eliminates, or reduces symptoms of pain or inflammation in the subject.

Example 9 relates to the method according to Example 8, wherein the hydrogel-forming composition further includes a crosslinking agent comprising a genipin crosslinking agent, a lysyl oxidase (LOX) crosslinking agent, or a combination thereof.

Example 10 relates to the method according to Example 9, wherein the crosslinking agent aids in forming inter-collagenous covalent bonds between the hydrogel-forming composition and adjacent intervertebral disc tissue of the subject.

Example 11 relates to the method according to Example 8, wherein the subject is a human, a dog, a cat, or a horse, and wherein the hydrogel-forming composition is administered to the subject via an injection.

Example 12 relates to the method according to Example 11, wherein the hydrogel-forming composition is administered to a joint of the subject, wherein the joint is a synovial joint, vertebral joint, temporomandibular joint, sacroiliac joint, finger or toe joint, ankle joint, elbow joint, hip joint, or a combination thereof.

Example 13 relates to the method according to Example 8, wherein the hydrogel-forming composition is administered into an intervertebral disc of the subject.

Example 14 relates to the method according to any one of Examples 8 to 13, wherein the hydrogel-forming composition is neuroinhibitory, and wherein the hydrogel-forming composition prevents nerve growth, increases intervertebral disc volume, increases intervertebral disc mechanics, or a combination thereof, resulting in the prevention, elimination, or reduction of symptoms of pain or inflammation in the subject.

Example 15 relates to the method according to Example 8, wherein the pain or inflammation in the subject is a result of low-back pain, neck pain, or a combination thereof.

Example 16 relates to the method according to Example 8, wherein the composition is administered to the subject as a single administration, or once every one to ten years.

In Example 17, a method of making a composition comprises digesting a decellularized nucleus pulposus tissue (dNP) to form a digested dNP via a treatment of the dNP with an enzyme; and combining the digested dNP with collagen to form a dNP gel.

Example 18 relates to the method according to Example 17, further comprising a step of combining a crosslinking agent comprising a genipin crosslinking agent, a lysyl oxidase (LOX) crosslinking agent, or a combination thereof to the dNP gel to form a hydrogel-forming composition.

Example 19 relates to the method according to Example 17, wherein the dNP is present in the composition in an amount of between about 1 mg/mL to about 15 mg/mL, wherein the enzyme comprises pepsin in a concentration of between about 1 mg/mL to about 10 mg/mL, and wherein the collagen comprises type I collagen in an amount of from about 1 mg/mL to about 9 mg/mL.

Example 20 relates to the method according to Example 17, wherein the digested dNP is further neutralized with a basic agent to result in a pH of between about 6 and about 9 prior to combining with the collagen.

While multiple embodiments are disclosed, still other embodiments of the present disclosure will become apparent to those skilled in the art from the following detailed description, which shows and describes illustrative embodiments of the disclosure. Accordingly, the figures and detailed description are to be regarded as illustrative in nature and not restrictive.

### BRIEF DESCRIPTION OF DRAWINGS

FIG. 1 shows a schematic illustration of the porcine nucleus pulposus decellularization, digestion, and re-gelation process and parallel analyses.

FIG. 2A is a graph showing DNA concentrations comparing before and after the decellularization process and after making into a gel. DNA was significantly reduced in the decellularized nucleus pulposus (NP) compared to control. * Indicates a significant difference at p<0.05.

FIG. 2B is a graph showing sGAG concentrations comparing before and after the decellularization process and after making into a gel. sGAG concentration was maintained in the decellularized NP compared to control. * Indicates a significant difference at p<0.05.

FIG. 2C is a graph showing collagen concentrations comparing before and after the decellularization process and after making into a gel. Collagen concentration was maintained in the decellularized NP compared to control. * Indicates a significant difference at p<0.05.

FIG. 2D is a graph showing the metabolic activity of NP cells treated with conditioned medium from decellularized NP tissue, normalized to untreated control shows a slight reduction to 86% of the control, which indicates minimal cytotoxic effects.

FIG. 3 is a graph showing both the control and decellularized NP had significantly fewer positive alpha gal epitopes compared to muscle. * Indicates a significant difference at p<0.05.

FIG. 4A is a graph showing the change in absorbance measured over time of collagen and re-gelled NP demonstrating a large and significant difference, suggesting that the re-gelled NP becomes more opaque than collagen.

FIG. 4B is a graph showing the normalized absorbance of the gels suggesting that collagen only reaches its maximum value much sooner than the re-gelled NP.

FIG. 4C is a graph showing rheology comparing the storage (G′) and loss (G″) moduli between collagen and re-gelled NP, suggesting that re-gelled NP is weaker than collagen. There were no significant differences between the G″ of either group but the G′ of collagen was significantly different than that of re-gelled NP at 69.5 and 100 rad/s. Error bars were omitted to improve clarity.

FIG. 4D is a graph showing rheology comparing the dynamic shear modulus (G*) and tan(δ) of collagen and re-gelled NP suggesting that re-gelled NP is weaker than collagen. G* of collagen was significantly different from that of re-gelled NP at 69.5 and 100 rad/s, while tan(δ) was significantly different at 23.4 rad/s. Error bars were omitted to improve clarity.

FIG. 5 is a graph showing the average fiber diameter in nm between Control NP, Decellularized NP, Collagen Only, and Regelled NP. There were no significant differences between any group.

FIG. 6A is a graph showing the PicoGreen DNA results demonstrating an increase in DNA from day 0 to day 7 in the regelled NP groups, while the collagen group decreased on day 3. * Indicates a significant difference at p<0.05.

FIG. 6B is a graph showing the AlamarBlue results showing a significant increase in metabolic activity of each group of gels normalized to DNA on day 7 in the regelled NP gels to all other time points. The regelled NP groups were also significantly greater than the collagen group on day 7. * Indicates a significant difference at p<0.05.

FIG. 6C is a graph showing the sGAG quantification results, revealing a significant decrease in the regelled NP groups from day 0 to the following days, however, all groups appeared to increase in sGAG from day 3 to day 7, although not significant. * Indicates a significant difference at p<0.05.

FIG. 6D is a graph showing the quantification of collagen content results, showing that the collagen was stable over time in both groups, with only day 0 and day 1 in the regelled NP group being significantly different. The collagen control group was significantly lower than the regelled NP at each time point. * Indicates a significant difference at p<0.05.

FIG. 7A is a graph showing the analysis of neurite growth into regelled NP inner gels compared to collagen inner gels. The graph shows the maximum neurite length in μm. The graph shows the average and standard deviation of the gels individually to show the scattering of the individual points.

FIG. 7B is a graph showing the analysis of neurite growth into regelled NP inner gels compared to collagen inner gels. The graph shows maximum radial distance into the inner gels. The graph shows the average and standard deviation of the gels individually to show the scattering of the individual points. * Indicates significant difference at p<0.05.

FIG. 7C is a graph showing the analysis of neurite growth into regelled NP inner gels compared to collagen inner gels. The graph shows the number of neurons at specific distances into the inner gel. * Indicates significant difference at p<0.05.

FIG. 8A is a diagram showing a proposed treatment for disc-associated pain using decellularized healthy NP tissue, collagen, and genipin to be tissue integrating, spontaneously fibrillogenic, cytocompatible, and biomechanically restorative leading to pain-like behavior remission.

FIG. 8B is a diagram showing the treatment process, including testing decellularized nucleus pulposus gels supplemented with 6.0 mg/mL collagen and genipin from 0-20 mM. Testing of these gels (dNPs) included gelation kinetics, rheology, cytotoxicity, and ex vivo capacity to restore injured disc mechanics.

FIG. 8C is a diagram showing how disc degeneration was induced in female Sprague Dawley rats and allowed to progress for 8 weeks. At 9 weeks post-injury, half of the injured animals were treated with dNP+(dNP+collagen+genipin) and the other half with 1×PBS.

Throughout the in vivo arm, disc volume and pain-like behavioral metrics were collected to monitor effects of disc injury and treatment.

FIG. 9A is a graph showing the absorbance of dNP gels with genipin at 405 nm over 12 hours of incubation at 37° C.

FIG. 9B is a graph showing absorbance of dNP gels with genipin at 405 nm within the first 20 minutes. The graph shows that all gels exhibited rapid fibrillogenesis in the first 20 minutes of incubation.

FIG. 9C is a graph showing absorbance of dNP gels with genipin at 610 nm over 12 hours of incubation at 37° C. As part of the crosslinking reaction, genipin forms genipin blue, which absorbs light at 610 nm. 20 mM genipin dNP gels were the first to exhibit a dramatic increase in absorbance although all genipin supplemented gels increased in absorbance at 610 nm. The final absorbance values for 10 mM and 20 mM dNP gels were similar, suggesting all crosslinking sites were exhausted at around 10 mM genipin. The final absorbance of 2.5 mM genipin was at the midpoint between 20 mM and 0 mM.

FIG. 9D is a graph showing absorbance of dNP gels with genipin at 610 nm within the first 4 hours. All gels exhibited genipin crosslinking initiation between 1:30:00 and 3:30:00 hours.

FIG. 10A is a graph showing the storage moduli of the dNP with various concentrations of genipin. These values represent the elastic resistance to torsional strain. The variability increased dramatically with genipin concentrations greater than 5 mM. Data is shown as mean with standard deviation. Gel fabrication was performed in duplicate in three separate experiments (n=3).

FIG. 10B is a graph showing the loss moduli of the dNP with various concentrations of genipin. These values represent the viscous resistance to torsional strain. Like the storage moduli, variability increased dramatically when genipin concentrations exceeded 5 mM. Data is shown as mean with standard deviation. Gel fabrication was performed in duplicate in three separate experiments (n=3).

FIG. 11 is a graph showing the motion segment storage moduli from undamaged, injured, and dNP+ treated motion segments. The average storage modulus of undamaged control motion segments did not change over the course of the experiment. Injury decreased the average storage modulus by 50%. Treatment with dNP+ significantly rescued the storage modulus by 15% compared to post-injury. Data is shown as mean with standard deviation (n=3 per group). Significance is denoted by *=p<0.05.

FIG. 12A is a graph showing the quantification of treatment cytotoxicity. All treatments exhibited minimal cytotoxicity with the lowest average viability occurring in the dNP+ treatment at 94.75%. Data is shown as mean with standard deviation. Experiments consisted of three unique donors plated in triplicate (n=3).

FIG. 12B is a graph showing the quantification of cell size. Cells plated on polystyrene had significantly larger average area than those plated on collagen+dNP. Data is shown as mean with standard deviation. Experiments consisted of three unique donors plated in triplicate (n=3). Significance is denoted by *=p<0.05.

FIG. 12C is a graph showing the quantification of total cell proliferation over the 48 hours of culture. No significant differences were detected between any treatment group. The average dNP+ treatment cell count was 66% that of collagen+dNP but this difference narrowly missed significance (p<0.052). Data is shown as mean with standard deviation. Experiments consisted of three unique donors plated in triplicate (n=3).

FIG. 13 is a graph showing L5-L6 intervertebral disc volumes calculated from uCT data. The disc volume in both dNP+ and PBS animals significantly decreased after injury compared to sham animals. After treatment surgery, dNP+ treated animal disc volumes returned to baseline and were no longer significantly different from sham. Data is shown as mean with standard deviation (n=11-12 per group). #=p<0.05 sham vs. dNP+. *=p<0.05 sham vs. PBS. &=p<0.05 dNP+vs. PBS.

FIG. 14 is a graph showing 15-week grip strength before and after treating with diclofenac. Diclofenac was effective at suppressing axial hypersensitivity as noted by the highly significant increase in animal grip strength after drug administration in PBS animals. (n=11-12 per group). Significance is denoted by ****=p<0.05.

Various embodiments of the present disclosure will be described in detail with reference to the figures. Reference to various embodiments does not limit the scope of the disclosure. Figures represented herein are not limitations to the various embodiments according to the disclosure and are presented for exemplary illustration of the disclosure.

### DETAILED DESCRIPTION

The embodiments of this disclosure are not limited to particular hydrogel-forming compositions, which can vary and are understood by skilled artisans. It is further to be understood that all terminology used herein is for the purpose of describing particular embodiments only and is not intended to be limiting in any manner or scope. So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1%, and 4% This applies regardless of the breadth of the range.

The term “about,” as used herein, refers to variation in the numerical quantity that can occur, for example, through typical measuring techniques and equipment, with respect to any quantifiable variable, including, but not limited to, mass, volume, temperature, and time. Further, given solid and liquid handling procedures used in the real world, there is certain inadvertent error and variation that is likely through differences in the manufacture, source, or purity of the ingredients used to make the compositions or carry out the methods and the like. Whether or not modified by the term “about,” the claims include equivalents to the quantities.

The term “actives” or “percent actives” or “percent by weight actives” or “actives concentration” are used interchangeably herein and refers to the concentration of those ingredients involved in cleaning expressed as a percentage minus inert ingredients such as water or salts. It is also sometimes indicated by a percentage in parentheses, for example, “chemical (10%).”

The term “pain” as used herein, refers to an unpleasant sensory and emotional experience associated with, or resembling that associated with, actual or potential tissue damage. Pain is part of the human experience and assists in weighing, alerting, avoiding and correcting actions that can lead to harm. Despite being variable and individualized, pain duration is diagnostically broken into two categories: acute and chronic. Acute pain is directly linked with tissue damage, inflammation, and the wound healing process. This type of pain is essential for evolutionary survival to avoid danger and facilitate healing. Chronic pain, or pain which outlasts the wound healing window, contains little evolutionary value, and can be viewed as a disease itself. While there is not complete consensus, generally, pain that exists past the expected healing period (˜three months in humans) is categorized as chronic pain.

The term “chronic low back pain” as used herein, refers to pain and discomfort, localized between the bottom of the ribs and the crease of the buttocks, with or without leg pain, that persists beyond three months. This type of chronic pain can range in intensity from a mild nuisance to complete debilitation.

The term “disc-associated pain” as used herein, refers to chronic pain associated with changes in, or arising from, the intervertebral disc.

The term “treating,” “treatment,” or “treat” as used herein, refers to any action, intervention, or administration that aims to alleviate, mitigate, ameliorate, manage, reduce the severity of, prevent the occurrence of, or cure a particular condition, disorder, disease, or symptom. Such actions may include, but are not limited to, the use of pharmaceutical agents, medical devices, surgical procedures, therapeutic techniques, lifestyle modifications, and any other methods intended to bring about a positive change in the health, well-being, or physiological state of a subject. The terms encompass both therapeutic and prophylactic interventions, and encompasses a range of degrees of improvement, from complete resolution to partial relief. It is to be understood that the terms “treating,” “treatment,” or “treat” as used herein do not imply absolute or guaranteed efficacy, and the outcomes may vary based on individual responses, underlying factors, and other variables.

The term “weight percent,” “wt. %,” “wt-%,” “percent by weight,” “% by weight,” and variations thereof, as used herein, refer to the concentration of a substance as the weight of that substance divided by the total weight of the composition and multiplied by 100.

All publications and patent applications in this specification are indicative of the level of ordinary skill in the art to which this disclosure pertains. All publications and patent applications are herein incorporated by reference to the same extent as if each individual publication or patent application was specifically and individually indicated as incorporated by reference.

Compositions

The present disclosure is generally directed to hydrogel-forming compositions and methods of making the hydrogel-forming compositions for use in methods of treating a subject having symptoms of pain or inflammation. In embodiments, the combination of the components disclosed herein form a robust hydrogel-forming composition capable of integrating with surrounding tissue around the site of administration when administered to a subject. In aspects, the hydrogel-forming compositions are neuroinhibitory, and the administration of the hydrogel-forming composition may result in a number of beneficial effects, including, but not limited to, preventing nerve growth, increasing intervertebral disc volume, increasing intervertebral disc mechanics, or a combination thereof, resulting in the prevention, elimination, or reduction of symptoms of pain or inflammation in the subject.

In some aspects, the pain or inflammation stems from a joint or surrounding tissue associated with nerve growth. In some embodiments, the pain is located in the spine, the back, the neck, the knee, or the jaw, including in a joint or surrounding tissue of a subject. In preferential embodiments, the pain is located in the spine, the back, or the neck. In embodiments, the joint may include, but is not limited to, a spinal motion segment, a synovial joint, vertebral joint, temporomandibular joint, sacroiliac joint, finger or toe joint, ankle joint, elbow joint, hip joint, or a combination thereof. In some embodiments, the pain or inflammation of the joint may further result in, but is not limited to, low back pain, neck pain, or a combination thereof. In further embodiments, the joint may include an intervertebral disc or cartilaginous endplate. In some aspects, the pain includes disc-associated pain, such as, pain stemming from the intervertebral disc (IVD).

The IVD is a cartilaginous tissue that acts as a joint between vertebral bodies of the spine and is predominantly aneural. Due to age, injury, environment, and/or genetics, cells of the IVD can become stressed, upending tissue homeostasis into catabolism. IVD catabolism is defined by a pro-inflammatory milieu consisting of cytokines, chemokines, and enzymes such as TNF-α, IL-1β, IL-6, CCL2, CCL5, MMPs, and ADAMTs. These factors recruit and activate immune cells and break down the extracellular matrix, resulting in inflammatory persistence and biomechanical decline. Coinciding with degeneration, a loss of neuroinhibitory proteoglycans and formation of annular fissures creates a neuro-permissive environment, allowing nerves to aberrantly penetrate the IVD. While the presence of nerves within degenerated IVD is the basis for pain, there is considerable evidence that altered biomechanics drives nociception, poising hypermobility as the primary causative agent in disc-associated pain. In aspects, spinal destabilization alone can produce disc-associated pain-like phenotypes in animals.

In some aspects, the IVD is composed of a gelatinous nucleus pulposus (NP) core surrounded by a lamellar annulus fibrosus (AF). The healthy disc is predominantly avascular and aneural and receives the majority of nutrients from diffusion through adjacent cartilaginous endplates. Aging and injury can trigger disc degeneration, resulting in thickening of the cartilaginous endplates, reducing diffusion of nutrients into the NP. This reduction in nutrient diffusion can lead to NP cell senescence. In aspects, senescent NP cells secrete inflammatory cytokines and degradative enzymes, creating a catabolic environment that breaks down matrix components, such as aggrecan. Intact aggrecan has neuroinhibitory properties due to sulfated glycosaminoglycan (sGAG) side chains and thus, naturally inhibits nerve growth into a healthy disc. However, during degeneration, cleavage of these sGAG side chains can reduce the neuroinhibitory properties of the disc. Further, in embodiments, senescent NP cells may secrete several molecules that increase nerve growth, such as nerve growth factor (NGF) and brain-derived neurotrophic factor (BDNF). The loss of the potently neuroinhibitory sGAGs and increased expression of NGF and BDNF result in ideal conditions for the ingrowth of nerve fibers into degenerated discs. In aspects, once painful nerve fibers are present in the disc, they can be sensitized and stimulated by the harsh catabolic environment of the degenerating disc, leading to pain. Treatments that contain neuroinhibitory sGAGs and other neuroinhibitory components have the potential to prevent disease progression by preventing nerve growth. Therefore, in some aspects, decellularized tissue scaffolds fabricated from healthy NP tissue can reintroduce neuroinhibitory properties to the NP and prevent pain progression.

In some aspects, any innervated structure of the low back may be a potential source of chronic low back pain. Observing that IVD is a strong associative factor of chronic low back pain, it also represents a therapeutic target with significant potential impact if treated. Chronic low back pain arising from the IVD is associated with many research and clinical terms, including, but not limited to, discogenic pain, degenerative disc disease, painful IVD degeneration (IDD), and disc-associated pain. In humans, disc-associated pain is notoriously difficult to diagnose, and may clinically manifest in a few different phenotypes. Axial low back pain is the most common type of chronic low back pain and is defined by pain that only localizes to the low back and occurs in a patient identifiable region on the body. Axial pain production is localizable, and the cause can often be estimated. This type of pain worsens with certain activities like running and even at rest if an incorrect postural position is maintained. Without being limited to any particular theory or mechanism, axial low back pain may originate in IVD tissue, arising from nerve fibers which aberrantly sprout into degenerated IVD tissue.

As further described herein, compositions comprising novel biomaterial are provided. In embodiments, the composition comprises nucleus pulposus tissue derived from decellularized, healthy, IVD material. In aspects, the composition is naturally fibrillogenic, cytocompatible, and neuroinhibitory. The composition may further comprise collagen and an optional crosslinking agent to create a robust hydrogel capable of integrating with degenerated IVD tissue. As further described herein, the compositions are cytocompatible and effective at restoring IVD volume, disc mechanics, and alleviating axial hypersensitivity post IVD degeneration.

In embodiments, the decellularized tissues provide successful compositions for the treatment of pain and inflammation in a subject because decellularized scaffolds have similar properties, compositions, and architecture to native tissue, thereby improving their function and cellular recognition in vivo compared to other treatments. In aspects of the clinical implantation of animal tissue, it is not feasible to implant animal tissue directly into a human subject, as doing so will cause a multitude of immune responses and be rejected quickly. Therefore, in aspects, decellularization may address the issue, as antigens from cells are removed in the decellularization process, leaving behind structural and other native molecules, such as, but not limited to, sGAGs.

In embodiments, the compositions comprise a decellularized nucleus pulposus tissue (dNP). While any dNP may be used, in embodiments, the NP for decellularization is derived from porcine or bovine species, due to their similarities in composition to human NP, as well as their availability due to raising livestock. In some embodiments, the dNP is derived from decellularized porcine nucleus pulposus tissue. In further embodiments, the dNP is a digested dNP. In some aspects, the digested dNP is formed by the treatment of dNP with an enzyme.

While any enzyme that can digest tissue may be used, in some embodiments, the enzyme comprises pepsin. In embodiments, the pepsin is present in a range of between about 1 mg/mL to about 10 mg/mL.

In even further embodiments, the digested dNP is further neutralized with a basic agent to result in a pH of between about 6 and about 9, between about 6.5 and about 8.5, or between about 7 and about 8. While any basic agent may be used, in embodiments, the basic agent may include, but is not limited to, sodium hydroxide, potassium hydroxide, calcium hydroxide, magnesium hydroxide, sodium carbonate, sodium bicarbonate, or a combination thereof.

Decellularization of the NP is critical, as the success of the immune response induced by a tissue scaffold can depend on the efficiency of decellularization. In examples, if a scaffold or biomaterial can guide the immune response to become pro-regenerative, it will have increased therapeutic efficacy, and increase the time that the biomaterial can function due to decreased degradation rate. In terms of immunomodulation for tissue engineering scaffolds, the key immune cell to target is the macrophage. During times of tissue damage or infection, macrophages are polarized towards an inflammatory phenotype, where they secrete inflammatory cytokines and chemokines in order to fight off infection and alert other cells of what is happening. This phenotype is termed the M1-like phenotype. During times of tissue remodeling and maintenance, macrophages can enter into an alternatively activated, pro-remodeling phenotype where they secrete anti-inflammatory cytokines and aid in the remodeling process through phagocytosis of damaged molecules. This phenotype is termed the M2-like phenotype. In some embodiments, the ideal macrophage response to a decellularized scaffold would be to polarize towards an M2-like phenotype, so that the macrophage can begin to remodel the scaffold accordingly. As provided within the disclosure, effective decellularization of NP tissue provides for a higher M2-like response. Therefore, in some aspects, it is important to achieve a high percentage of decellularization of the NP tissue in preparing the dNP of the compositions and methods provided herein. In embodiments, the dNP has at least a 70% reduction in DNA, at least an 80% reduction in DNA, at least a 90% reduction in DNA, at least a 95% reduction in DNA, or at least a 98% reduction in DNA.

In embodiments, the dNP is present in the composition in an amount of between about 1 mg/mL and about 20 mg/mL, between about 1 mg/mL and about 19 mg/mL, between about 1 mg/mL and about 18 mg/mL, between about 1 mg/mL and about 17 mg/mL, between about 1 mg/mL and about 16 mg/mL, or between about 1 mg/mL and about 15 mg/mL.

In further embodiments, the hydrogel-forming compositions contain collagen. Collagen is the principal extracellular structural protein in a mammal. At least seven types of mammalian collagen have been described. Their common characteristic is a three stranded helix, consisting of 3 polypeptide chains, called alpha-chains. All of the alpha chains have the same configuration but differ in the composition and sequence of their amino acids, leading to different types of alpha chains. However, the chains all have glycine in every third position of the amino acid sequence, allowing the helical conformation to occur. In the compositions of the disclosure, the collagen may be added to the dNP to form a dNP gel or hydrogel-forming composition. Throughout the present disclosure, “dNP gel” may be further referred to herein as “re-gelled NP” or “re-gelled decellularized NP tissue”. In some cases, the collagen is added to dNP that has already been digested.

Representative collagen materials include rat tail collagen, placental collagen, recombinant human collagen, tissue engineered human-based collagen, porcine collagen, bovine collagen, autologous collagen, collagen fibers, and human tissue collagen matrix. Collagen's structural and functional properties are uniquely suited to these diverse applications. For example, collagen can be useful in tissue engineering procedures in which an implanted device serves to guide proper tissue regeneration, providing structural support and a suitable surface for cell and tissue growth/regrowth. Collagen's absorbable properties minimize the likelihood of infections and other downstream adverse immunological reactions associated with the implanted material. Further, collagen is hemostatic, making it suitable for use in medical sponges, bandages, dressings, sutures, etc. Collagen facilitates wound healing, tissue regeneration, etc., by providing sites for cell attachment and migration. Collagen's three-dimensional structure permits effective drug and nutrient exchange with the surrounding environment and prevents build-up of waste products, etc., enabling its use in various drug delivery devices and systems, facilitating cell/tissue growth/regrowth in engineering applications, etc.

Collagen or collagen-containing biomaterials can include collagen extracted from a variety of sources especially naturally derived animal tissues, such as human tissue. In other embodiments, the collagen in the collagen composition is entirely obtained through non-natural, or synthetic sources. In further embodiments, collagen or collagen-containing tissues are a mixture of collagen derived or obtained from more than one source.

Any type of collagen may be used in the methods and compositions herein. In some embodiments, type I collagen, type II collagen, or a combination thereof, may be used. In some preferred embodiments, the collagen is type I collagen. The collagen may be derived from cell culture, animal tissue, or recombinant means, and may be derived from human, porcine, or bovine sources. Some embodiments comprise collagen derived from human fibroblast culture. Some embodiments comprise collagen that has been denatured to gelatin.

In embodiments, the dNP is present in the composition in an amount of between about 1 mg/mL and about 15 mg/mL, between about 1 mg/mL and about 14 mg/mL, between about 1 mg/mL and about 13 mg/mL, between about 1 mg/mL and about 12 mg/mL, between about 1 mg/mL and about 11 mg/mL, between about 1 mg/mL and about 10 mg/mL, or between about 1 mg/mL and about 9 mg/mL.

In some embodiments, the hydrogel-forming compositions provided herein may optionally include a crosslinking agent. In some aspects, the inclusion of a crosslinking agent may modify the mechanical properties of the hydrogel-forming composition. Beneficially, the inclusion of a crosslinking agent may assist in forming inter-collagenous covalent bonds between the hydrogel-forming composition and degenerated tissue surrounding a site of injection. In some aspects, the administration of the hydrogel-forming compositions to a subject will result in a hydrogel composition upon cross-linking between the crosslinking agent (such as genipin, for example) and degenerated tissue surrounding the site of injection. In some embodiments, the degenerated tissue surrounding the site of injection is degenerated disc tissue.

While a variety of crosslinking agents may be used, examples of suitable crosslinking agents for use within the hydrogel-forming compositions of the disclosure include genipin, lysyl oxidase (LOX), or a combination thereof. Genipin is a naturally occurring compound extracted from the fruiting bodies of Gardenia Jasminoides. At concentrations below 20 mM, genipin remains cytocompatible and can dramatically increase the mechanical properties of biomaterials derived from amino acid rich components like elastin, gelatin, and collagen. In aspects, genipin contains multi-active functional groups and can perform cross-linking reactions with a macromolecular material containing amino functional groups in a single-molecule or multi-molecule form. Compared with traditional crosslinking agents, genipin provides good biocompatibility and certain anti-inflammatory activity, can inhibit the release of inflammatory factors and metal matrix protease, and has unique advantages when being used in wound treatment materials. When added to tissues or hydrogels rich with collagen, genipin molecules can react with lysine residues to form inter and intrafibrillar covalent crosslinks. The crosslinking process is relatively stochastic with higher genipin concentrations beginning crosslink formation earlier than lower concentrations.

In embodiments, where a crosslinking agent is included, the crosslinking agent may comprise genipin in an amount of between about 0.1 mM to about 20 mM, or between about 0.5 mM to about 20 mM. In further embodiments, where the crosslinking agent comprises LOX, the LOX crosslinking agent may be present in the composition in an amount of between about 0.02 to about 2 ng/mL, or between about 0.02 ng/mL to about 1 ng/mL. In some embodiments where LOX is used, the composition may further include copper (II) sulfate (CuSO₄). In aspects, the copper (II) sulfate may aid in activation of the LOX crosslinking agent.

In embodiments, the hydrogel-forming compositions have a neutral pH. For example, the pH of the compositions may be in the range of between about 6 and about 8, between about 6.5 and about 8, or between about 7 and 8. pH ranges falling outside of this range may further be possible.

In embodiments, the compositions may further include a pharmaceutically acceptable carrier or solvent. In some examples, suitable aqueous carriers, diluents, solvents, or vehicles may be used. In some aspects, the compositions may further include water, phosphate buffered saline (PBS), or a combination thereof. In some embodiments, the PBS may be 10×PBS, or a dilution thereof to 1×PBS in solution. In embodiments, the water may be deionized water, distilled water, or double-distilled water. In some aspects, the water is double-distilled water.

Methods

In embodiments, the method may include a step of diagnosing pain in a subject. In embodiments where the subject is experiencing disc-associated pain, clinical features such as, but not limited to, deep-seated and dull aching back pain, referred pain, decreased physical activity, and restricted spinal movement, have been viewed as important characteristics of disc-associated pain, and often as first line indicators a subject is experiencing disc-associated chronic low back pain.

In aspects of diagnosing pain in a subject, the subject may be asked to complete various pain, functional, and IVD morphological assessments to ascertain a diagnosis. The first and most important data collected at the clinic are body maps which allow a subject to describe the location and duration of pain. Body maps have become increasingly important in the last few years with the emphasis on individualized treatment. In embodiments, subjects with low back pain may be asked to complete questionnaires which measure various impacts of the pain to assess both the intensity and location of pain and how the pain affects life activities like socialization, employment, etc. Functional tests employ static and dynamic measures of trunk muscle activation during movement to quantify trunk strength, flexibility, and endurance. These aspects of the trunk are measured because they are known to be impaired for many subjects with chronic low back pain. The most widely used method for evaluating patient IVD morphology in the clinic is imaging via MRI. This assessment is performed to determine if spinal changes, especially IVD degeneration, are present and could be a source of pain. MRI images are used to determine degenerative changes in the IVD by identifying abnormalities like a loss of nuclear signal, decreased disc height, high-intensity zones, and changes in disc contours.

The methods as provided herein further include a method of treating a subject. In some aspects, the subject being treated is experiencing symptoms of pain or inflammation as described herein. In embodiments, the method of treating comprises administering the hydrogel-forming composition of the disclosure to a subject. In some aspects, the composition may be administered to a joint of a subject. The administration of the composition to the subject may eliminate or reduce symptoms of pain or inflammation in the subject. In some embodiments, the administration of the composition restores joint damage, restores tissue damage, increases intervertebral disc volume, increases intervertebral disc height, or a combination thereof, resulting in the elimination or reduction of symptoms of pain or inflammation in the joint of the subject. In further embodiments, the administration may further prevent symptoms of pain or inflammation in the subject.

The subject being treated may be a mammal. In embodiments, the subject may be a human or an animal. In embodiments wherein an animal is treated, the animal may include, but is not limited to, a dog, a cat, or a horse. In some aspects, the subject being treated is a human.

In embodiments, the composition is administered via an injection. In some aspects, the injection is via an ultrasound guided injection. In further embodiments, the composition may be administered to a subject through open surgery. In further embodiments, the composition of the disclosure is injected to a joint, surrounding tissue, or to an intervertebral disc of the subject. In some aspects, the hydrogel-forming composition for injection is made from digested (such as via enzymatic digestion) and re-gelled decellularized NP tissue. The digestion and regelation allow for the reforming of a gelatinous structure, similar to native NP, in situ. In some embodiments, the hydrogel-forming composition is in liquid form. In further embodiments, the hydrogel-forming composition begins forming a hydrogel after the dNP and collagen are combined. In some aspects, the hydrogel-forming composition will begin forming a hydrogel under neutral pH (or at a pH of between about 6 and about 8), and at a temperature of 35° C. or greater. In some embodiments, a hydrogel composition will begin to form at a temperature of 37° C. or greater. In further embodiments, the hydrogel-forming compositions may begin forming a hydrogel at temperatures less than 35° C., however, at a slower rate.

In embodiments wherein a crosslinking agent is included, the crosslinking agent of the hydrogel-forming composition may aid in forming inter-collagenous covalent bonds between the hydrogel-forming composition and tissue surrounding the joint of the subject after administration. In some aspects, the administration of the hydrogel-forming composition to a subject initiates the formation of a hydrogel composition upon cross-linking between a crosslinking agent and the tissue surrounding the joint of the subject, or upon thermal gelation. In further embodiments, after the dNP, collagen, and a crosslinking agent are combined, the hydrogel-forming composition will begin to develop into a hydrogel. The hydrogel forming process will take time, however, may be administered to a subject prior to developing completely into a hydrogel. In embodiments, the hydrogel-forming composition may be administered to a subject prior to developing into a hydrogel.

In aspects, injectable hydrogel-forming compositions are suitable for the methods provided herein, because they are non-invasive, easily targetable, and the gels form to the space that they are injected into. Injectable hydrogel-forming compositions are further an attractive form of treatment as they can be chemically modified and optimized to include specific drugs or cells, and the mechanical properties can be tuned to what is specifically needed for certain implementations.

As will be understood by those skilled in the art, any treatment regimen may be used depending on the type of pain, severity, and other individual factors associated with the subject being treated. In some aspects, the volume of the composition administered to a subject may depend on the volume of area being treated. For example, the administration volume may depend on the disc volume being treated in the subject, however, may be administered up to the total volume of the nucleus pulposus of each subject being treated (i.e., the inner gel of the intervertebral disc). In further embodiments, the composition may be administered to the subject as a single administration or may be administered once every one to ten years. In preferential embodiments, the treatment will be administered as little as possible to achieve relief to the subject. In some embodiments, this may be one to two times within the lifetime of the subject. In other embodiments, the treatment may need to be administered more often. In examples, the treatment may be administered for a total of two to twenty years, or as needed based on the needs of the subject.

In embodiments, the hydrogel-forming composition of the disclosure is neuroinhibitory. In some embodiments, reintroducing a neuroinhibitory gel into a subject has the potential to stop further ingrowth of nerves and halt the progress of disc-associated low back pain. The removal of cellular DNA is crucial to the decellularization process to prevent an immune response when applied in vivo. It is speculated that during degeneration, there is infiltration of immune cells, which can recognize foreign cellular components and cause a host to reject the implanted material. Thus, the methods as provided herein include a decellularization process to remove more than about 90%, about 95%, or about 99% of the original DNA from the NP tissue, to increase the ability of a host subject accepting the decellularized tissue in the future. Due to the high removal of DNA and lack of alpha-gal found within the decellularized NP, it is unlikely that the NP tissue will cause an immune response due to species differences when implanted in a subject.

In some examples, porcine tissue has had clinical success previously with dermis (Fortiva) and small intestinal submucosa (Cook Biotech), suggesting that porcine NP may be implanted without problem after decellularization. Besides DNA removal, the other important aspect of decellularization includes the maintenance of native proteins. In the NP, the majority of the extracellular matrix is composed of sGAGs and type II collagen. The decellularization processes of the disclosure aim to maintain as much of the native sGAGs and collagen as possible to maintain the neuroinhibitory and structural capabilities of the tissue. In some embodiments, the sGAG maintenance is greater than about 60%, about 65%, or about 70%. In some embodiments, the sGAG maintenance is around 74.0%, which is comparable to other NP decellularization processes which have a range of retentions between 55-97%.

In embodiments, methods of making the hydrogel-forming compositions as described herein are further provided. In aspects, the method comprises combining the dNP and collagen to form a dNP gel. A crosslinking agent may optionally be added to the dNP gel to form a hydrogel-forming composition. In aspects, the dNP is digested via the use of an enzyme that can digest tissue. In some embodiments, the enzyme may be pepsin. In even further embodiments, the digested dNP is further neutralized with a basic agent to result in a pH of between about 6 and about 8.5, between about 7 and about 8, or between about 7 and about 7.8. While any basic agent may be used, in embodiments, the basic agent may include, but is not limited to, sodium hydroxide, potassium hydroxide, calcium hydroxide, magnesium hydroxide, sodium carbonate, sodium bicarbonate, or a combination thereof.

### EXAMPLES

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

### Example 1

### Injectable Decellularized Nucleus Pulposus Tissue Exhibiting Neuroinhibitory Properties

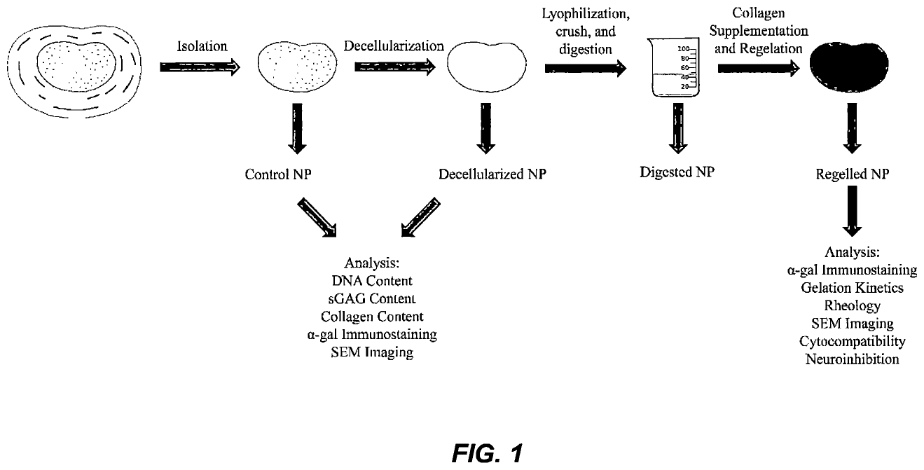

The cytotoxicity and neuroinhibitory properties of a dNP gel were evaluated using primary sensory neurons. The process involved whole disc decellularization, analyzing DNA content, analyzing sGAG content, analyzing collagen content, measuring the cytotoxicity of residual chemicals, conducting α-galactose epitope immunostaining, and the digestion and preparation of the decellularized NP gel. The dNP gels were then further evaluated for gelation kinetics, a mechanical characterization, and formation of collagen fibers. The NP cell cytocompatibility and neuroinhibitory properties of re-gelled NP were then evaluated. A schematic illustration of the process can be seen in FIG. 1.

### Methods

Whole Disc Decellularization—Cervical spines from commercial line Landrace/Yorkshire/Duroc young female pigs (˜200 days of age) were aseptically collected and frozen (−80° C.) following humane slaughter. Intact spines were thawed for two days at 4° C. The spines were then cleaned aseptically and the NPs of the C2-C7 intervertebral discs were surgically removed. Control NPs from each spine were either: 1) fixed in 4% paraformaldehyde (PFA) for 30 minutes, followed by 3×15 m washes in 1× phosphate buffered saline (PBS) for imaging, 2) eluted in media for cytotoxicity studies, or 3) frozen at −80° C. and lyophilized for 2 days (FreeZone 4.5 L Freeze Dryer (7750020, Labconco)) for additional analyses. The remaining NPs were decellularized following the process as outlined in Table 1.

TABLE 1

| | |
|---|---|
| Wash liquid | Time |

| | | | |
|---|---|---|---|
| 1 | ddH₂0 | 7 | hours |

| | | |
|---|---|---|
| 2 | 100 mM Sodium/50 mM Phosphate buffer | Overnight (10-12 hours) |

| | | | |
|---|---|---|---|
| 3 | 125 mM SB3-10 in 50 mM Sodium/10 mM | 4 | hours |
| | Phosphate buffer | | |
| 4 | 100 mM Sodium/50 mM Phosphate buffer | 15 | minutes |
| 5 | 3% (w/v) SD/0.6 mM SB-16 in 50 mM | 1 | hour |
| | Sodium/10 mM Phosphate buffer | | |
| 6 | 100 mM Sodium/50 mM Phosphate buffer | 3 × 15 | minutes |
| 7 | 125 mM SB3-10 in 50 mM Sodium/10 mM | 1.75 | hours |
| | Phosphate buffer | | |
| 8 | 100 mM Sodium/50 mM Phosphate buffer | 15 | minutes |
| 9 | 3% (w/v) SD/0.6 mM SB-16 in 50 mM | 45 | minutes |
| | Sodium/10 mM Phosphate buffer | | |
| 10 | 100 mM Sodium/50 mM Phosphate buffer | 3 × 15 | minutes |
| 11 | DNase (75 U/mL) in 50 mM Sodium/10 mM | 34 | hours |
| | Phosphate buffer | | |
| 12 | 50 mM Sodium/10 mM Phosphate buffer | 3 × 90 | minutes |
| 13 | 1X PBS | 3 × 3 | hours |
| 14 | ddH₂O | 3 × 15 | minutes |

Chemical Information for Decellularization Process
3-(Decyldimethylammonio)-propanesulfonate inner salt (SB3-10) (D4266, Sigma-Aldrich)
3(N,N-Dimethylpalmitylammonio)-propane inner salt (SB-16) (H6883, Sigma-Aldrich)
Sodium deoxylcholate (SD) (D6750, Sigma-Aldrich)
DNase (D4527, Sigma-Aldrich)

Decellularized NPs were processed as outlined in Table 1 with either fixation, elution, or lyophilization. A minimum of three spines were used for each analysis to account for animal variability. To account for increased variability in decellularized NP samples compared to control NP samples, at least one unprocessed control NP and at least one decellularized NP were analyzed for each spine, for a total of at least three control NPs and decellularized NPs.

DNA Content, sGAG Content, and Collagen Content—DNA content of control and decellularized NP samples was analyzed using the Quant-iT PicoGreen dsDNA Assay Kit (P7589, Thermo Fisher) according to the manufacturer's instructions to verify removal of antigenic DNA remnants after decellularization. Further, sGAG content of control and decellularized NP samples was quantified to determine maintenance of neuroinhibitory components after decellularization. Maintenance of collagen was determined after decellularization by analyzing collagen content in control and decellularized NP samples. A hydroxyproline assay (ab222941, Abcam) was conducted according to manufacturer's instructions with slight modifications to determine collagen content. Briefly, lyophilized control and decellularized NP samples were digested in 1 mL of 16 U/mL papain at 65° C. overnight. This digested NP was lyophilized overnight to concentrate the tissue. The resulting lyophilizate was hydrolyzed with 200 μL of 5 M sodium hydroxide (NaOH) at 120° C. for 1 hour, then neutralized on ice with 200 μL of 5 M hydrochloric acid (HCl). The remainder of the assay was conducted per the manufacturer's instructions and the resulting data was normalized to the dry weight of the tissue. A total of n=4 control NPs and n=4 decellularized NPs were used for the outlined experiments.

Cytotoxicity of Residual Chemicals—Cytotoxicity of any residual chemicals in the decellularized NP tissue was evaluated by measuring the change in metabolic activity of human NP cells treated with media eluted from the decellularized NP using the AlamarBlue Assay (Thermo Fisher, 88951) in accordance with ISO Standards 10993:5 and 10993:12. The alamarBlue assay measures the metabolic activity of cells by reducing resazurin in the electron transport chain to resoruflin. This reduction causes a change in color from blue to purple/pink that can be measured. Cells that are more metabolically active will reduce resazurin at a faster rate than less metabolically active cells, leading to a greater color change. All samples including untreated controls were measured in triplicate. A total of n=3 decellularized spines, with 4 NP samples taken from each n, for a total of 12 decellularized NP were used in the outlined experiments.

α-galactose Epitope Immunostaining—The α-galactosyl epitope (α-gal) content of decellularized NP scaffolds was tested because α-gal is known to elicit an immune response in humans. Immunostaining was used to test whether the α-gal epitope was present in the NP tissue after decellularization. Fixed control and decellularized NP samples were utilized for α-gal immunostaining. Porcine muscle tissue was used as an additional positive control for α-gal due to its high cellularity and high α-gal presence. Images were taken using a Zeiss Axio Observer at 10× magnification and quantified by counting the number of positive α-gal epitopes in three 10× images of control NP, decellularized NP, and muscle tissue from three different animals using ImageJ. A total of n=3 samples were used for each group with a minimum of 3 images analyzed per n.

Digestion and preparation of decellularized NP gel—The intact AF provides a barrier to delivery to the NP; thus, an injectable formulation is essential. To create an injectable gel made from decellularized NP tissue, previous enzymatic digestion protocols were adapted. 20 mg of lyophilized decellularized NP was digested by 1 mg/mL of pepsin (P6887, Sigma-Aldrich) in 0.05 N HCl for 44 hours with spinning at 300 rpm using a magnetic stir bar. Following digestion, all steps of making the re-gelled NP were performed on ice, until incubation at 37° C. HEPES buffer (H0887, Sigma-Aldrich) was added to attain a concentration of HEPES of 7.5 mM. The digest was then neutralized with 5 M NaOH to pH ˜7.4. The digested NP tissue alone did not form a robust gel at 37° C., so type I collagen (354249, Corning) was supplemented to create a more robust gel. Type I collagen was selected because there is an increase in type I collagen with aging and degeneration forms a gel more consistently than type II collagen. Since the goal of this material was not to regenerate a healthy disc, but rather to act as a neuroinhibitory supplement, collagen type will not impact this property. DMEM (D2429, Sigma-Aldrich) was used to control the ionic strength of the solution, as ionic strength has been demonstrated to have a strong effect on the gelation time, number of fibrils, and size of fibrils of collagen. The neutralized tissue digest, as described here after it had been neutralized, was added to pre-mixed tubes containing DMEM and HEPES so that the final concentration of DMEM would be 0.5× and HEPES would be 7.5 mM. Collagen was added last up to a concentration of 2.5 mg/mL. The final pH of the resulting solution was adjusted to ˜7.4 using 0.25M NaOH and the resulting ionic strength was ˜0.148 M. The final formulation with supplemented collagen was termed re-gelled NP (or further described herein as dNP gel).

Gelation Kinetics—The gelation kinetics (how much the gel absorbed light over time due to fibril formation), and gelation time (at what time the absorbance starts and stops changing), were investigated. Gel absorbance was used as an indicator of collagen fibrillogenesis. At a wavelength of around 4.5 nm, the absorbance of the gel depends on the amount of collagen fibril formation. The change in absorbance from the initial value and the normalized absorbance, (Equation 1), were calculated to show the percent of the maximum absorbance. A total of n=3 different preparations were used with 3 samples analyzed for each n.

$$NA = \frac{{A}_{2} - {A}_{1}}{Max - {A}_{1}} \qquad Equation 1$$

NA=normalized absorbance
A₂=absorbance at a specific time
A₁=initial absorbance
Max=maximum absorbance value over the time course

Mechanical Characterization—Mechanical characterization including rheology of the gel was performed and compared to previously published data on degenerated NP tissue: a linear compressive modulus of ˜5.3 kPa and a complex shear modulus at 1 rad/s of ˜17 kPa. Rheology testing was performed on gels using an Anton Paar MCR 302 with sand blasted plates and a humidity bath. N=8 collagen control experimental replicates and 3 re-gelled NP groups, each with 6 replicates, for a total of 18 re-gelled NP samples.

Scanning electron microscopy—Scanning electron microscopy (SEM) was performed to assess formation of collagen fibers after NP digestion and re-gelation. Fixed control NPs, decellularized NPs, and re-gelled NPs were processed for SEM using a graded dehydration method. ImageJ was used to quantify the fiber diameter by drawing a line across each distinguishable fiber and measuring the distance of the line. A total of n=3 different samples were characterized and at least 3 images per sample were analyzed for each group at 100,000× magnification to quantify fiber diameter.

NP Cell Cytocompatibility—NP Cell Culture—Human NP cells (4800, ScienCell) were cultured in a T75 flask (CLS430641U, Sigma Aldrich) coated with 15 μg of Poly-L-Lysine (0413, ScienCell). NP cells were cultured in Complete Nucleus Pulposus Cell Media (4801, ScienCell) which was changed every two to three days. The NP cells were grown to confluency in hypoxia (3.5% oxygen, 10% CO₂, 86.5% N₂) in a modular incubator chamber (MIC-101, Billups-Rothenberg) before being used. Cells from passages 2 and 3 from multiple freeze downs were used in these experiments. These cells were acquired from single, fetal donors and the identity of the cells was verified by the company through immunofluorescence with antibodies for fibronectin and vimentin (ScienCell).

3D Cell Culture within Re-gelled NP or Collagen Control Gels—NP cells were added to non-crosslinked re-gelled NP or collagen control solutions to attain a cell concentration of 2 million cells/mL. 30 μL of the gel and cell suspension was transferred to a sheet of parafilm to prevent sticking, then placed in the incubator at 37° C. for 40 minutes to crosslink before being transferred individually to 48-well plates. 400 μL of complete NP cell media was added to each well. Media was replaced every 2-3 days over the course of 7 days. An alamarBlue metabolic assay (88951, Thermo Fisher Scientific) was conducted on days 0, 1, 3, and 7 after initiation of culture to determine cell health. At the desired time point, alamarBlue reagent was added to each well at a 1:10 ratio and the plates were incubated at 37° C. in hypoxia for 3 hours. 200 μL of media from each well was moved in duplicate to a 96 well plate. The wells were then rinsed with 1×PBS before adding 400 μL of complete NP media and placing the plates back in the incubator under hypoxia. The absorbance of the removed media was analyzed using a microplate reader at 570 and 600 nm. The reduction of alamarBlue was calculated according to manufacturer's instructions. The reduction was then normalized to the DNA content of the respective wells, following the conclusion of the experiment. Gels were then frozen in water at −80° C. at respective time points for sGAG, collagen, and DNA assays. A total of n=6 experimental replicates from each preparation, with the collagen having 1 preparation and the re-gelled NP having three different preparations for a total of 6 collagen and 18 re-gelled NP. At study end, all gels were lyophilized overnight. Due to the small masses remaining following lyophilization, the mass could not accurately be determined for normalization. Instead, the data was normalized to each gel, which was a known volume of solution. Three gels from each time point and group were digested in 1 mL of 16 U/mL papain overnight at 65° C. sGAG, DNA, and hydroxyproline assays were conducted as described previously in the “Validation of Whole Disc Decellularization Methods” section. A total of n=3 experimental replicates from each preparation, with the collagen having 1 preparation and the re-gelled NP having three different preparations for a total of 3 collagen and 9 re-gelled NP.

Evaluation of Neuroinhibitory Properties of Re-gelled NP—Dorsal Root Ganglia Explant Isolation Process—Studies using primary rat dorsal root ganglia (DRG) explant culture tested the neuroinhibitory properties of the re-gelled NP. DRGs are located close to the disc and can sprout pain-sensing neurons into degenerate disc, leading to pain, thus they are an ideal model cell type to validate neuroinhibition in vitro. All animal experiments were conducted in accordance with the Guide for the Care and Use of Laboratory Animals and approved through the University of Nebraska-Lincoln's Institutional Animal Care and Use Committee (IACUC). Adult male Sprague Dawley rats (CD Rat 001, Charles River) aged 11-15 weeks were euthanized and L1-L6 DRGs were surgically removed and placed in cold trimming media: Neurobasal-A media (10888022, Thermo Fisher Scientific) with 10% Fetal Bovine Serum (FBS) (26140079, Thermo Fisher Scientific), 1% GlutaMax (35050061, Thermo Fisher Scientific), 1% Penicillin/Streptomycin (15140122, Thermo Fisher Scientific) and 2% B-27™ Plus Supplement (A3582801, Thermo Fisher Scientific). DRGs were transferred into a 60 mm petri dish with cold trimming media and excess tissue around the DRG was trimmed using surgical spring scissors under a stereo microscope (Stemi 508, ZEISS). Trimmed lumbar DRGs were divided into two or three equally sized pieces before culture.

Gel-within-gel Fabrication and DRG Explant Culture—To accurately model the growth of DRG neurons into a degenerative disc in vitro, the previously established gel-within-gel 3D culture method was used to model the in vivo environment of the DRG and NP. Cut DRGs were embedded and cultured in type I collagen outer hydrogel which surrounded an inner ‘NP-like’ gel. Inner gels consisted of either re-gelled NP or type I collagen. Collagen inner gels was used as a positive control due to its neuro-permissive properties. To prepare the model, re-gelled NP and collagen gels were prepared as previously described herein with a final collagen concentration of 3.6 mg/mL, to match the final re-gelled NP collagen concentration which equated to the summation of the added type I collagen (2.5 mg/mL), and the collagen from the digested tissue (1.1 mg/mL). To form the inner gels, 100 μL of non-crosslinked collagen control or re-gelled NP were transferred into a 96 well plate with custom laser-cut plastic gel lifters and incubated for 60-90 minutes at 37° C., 5% CO₂. The inner gels were carefully lifted out and placed into a 48 well plate using the gel lifters. On ice, 150 μL of the prepared collagen outer gel was then pipetted to the outside of the inner gel, and a DRG explant was carefully embedded in the outer gel, close to the boundary between the two gels. The gel-within-gel was then incubated at 37° C., 5% CO₂ and 95% air for 1 hour to induce collagen gel crosslinking, prior to media addition. The DRGs were cultured in complete media: Neurobasal-A media with 10% FBS, 1% GlutaMax, 1% Penicillin/Streptomycin, 2% B-27™ Plus Supplement and 0.05% Nerve Growth Factor (NGF) (556NG100, Thermo Fisher Scientific) at 37° C. and 5% CO₂ for 15 days with half media changes every three days. This experiment was repeated three times with at least three inner gels per group. DRGs without any substantial neurite outgrowth in any direction after 15 days in vitro were excluded from this study.

Neuroinhibition Analysis—A total of three experiments with 11 total collagen controls and 20 re-gelled NP gels were imaged and analyzed. On day 15, DRGs were fixed in 300 μL of 4% PFA for 1 hour at room temperature. DRGs were then washed 3×15 minutes with 1×PBS and stored in 1×PBS at 4° C. protected from light until immunostaining. Fixed DRGs in gels were stained for neurofilament-H (NF-H). Briefly, the gels were permeabilized in 200 μL of blocking buffer (0.5% Triton X-100, 4% goat serum in 1×PBS) for 1 hour at room temperature. The blocking buffer was removed and 200 μL of primary antibodies against NF-H (mouse α-NF-H, Ab528399, DSHB) (1:500 in blocking buffer) were added to each well for 36 hours at 4° C. The primary antibody was removed, and the gels were washed 3×4 hours in PBST (0.05% Tween 20 in 1×PBS) with mild agitation at room temperature. Secondary antibody (α-mouse 488, ab150117, Abcam) (1:500 in blocking buffer) was added and incubated overnight at 4° C., protected from light. The secondary antibody was removed, and the gels were washed 3×4 hours in PBST at room temperature, with mild agitation, protected from light. The gels were stored in 1×PBS at 4° C., protected from light until imaged. Following staining, the gels were imaged using a widefield fluorescence plate imager (Cytation 1, Biotek) at 488 nm to visualize the neurons. Representative images were taken on a ZEISS Confocal microscope LSM 800. Brightfield photos of the gel-within-gels were taken using a digital camera (EOS Rebel T6i, Canon) attached to a stereomicroscope (Stemi 508, Zeiss).

Using Adobe Photoshop CC 2019, the brightfield images were overlayed over the fluorescent images taken from the Cytation plate imager and gel boundaries were drawn. Gels without distinct boundaries were excluded from analysis. ImageJ software, Simple Neurite Tracer tool, and a modified Sholl analysis were used to quantify the: 1) maximum radial distance, 2) total distance travelled in the inner gel, 3) number of neurites extending within the inner gel at specific distances from the gel boundary, and 4) the number of neurites extending away from the inner gel at specific distances from the DRG body. The maximum radial distance was determined by drawing a straight line the tip of the longest neurite to where it initially crosses the gel boundary. The maximum neurite length was determined by tracing the longest nerves using the simple neurite tracer tool up until the gel boundary. Only the largest values were used from each image for both the radial distance and neurite length. Sholl analysis has previously been used to determine the amount of neurite branching based on distance from the cell body. This method was modified in this Example to represent distance from the boundary of the inner and outer gels, as well as the number of neurites growing away from the DRG body. Sholl analysis was able to determine the number of neurites at a given distance from the gel boundary. A total of n=3 experiments were conducted with at least 3 replicates per condition in each experiment.

### Results

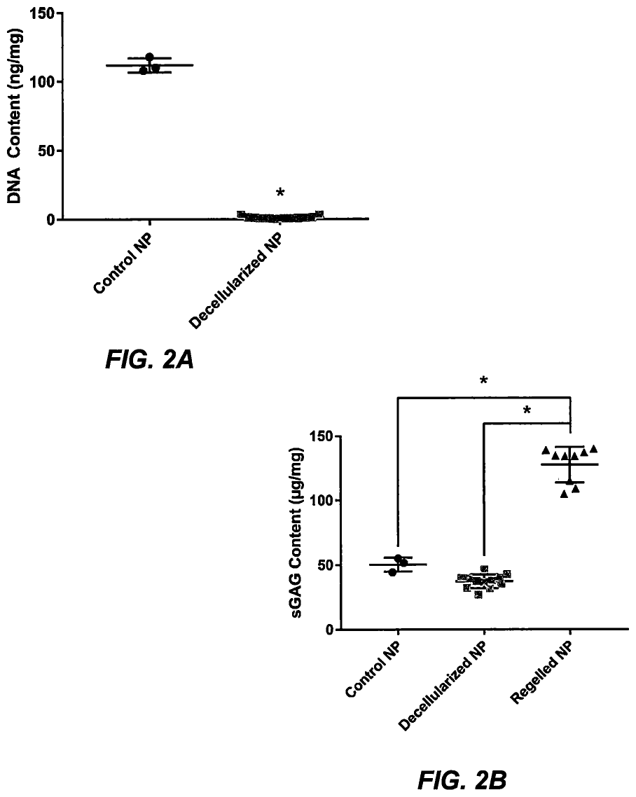

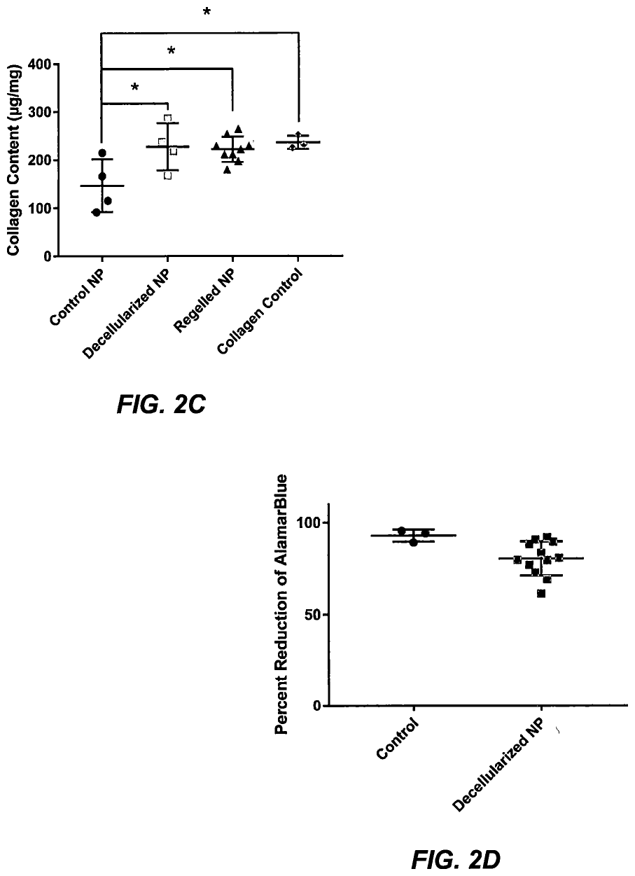

Validation of Whole Disc Decellularization Methods—The PicoGreen assay demonstrated 99.0% reduction in DNA in the decellularized NP samples compared to the control NP tissue, with the decellularized NP samples having a normalized DNA content of 1.19±0.94 ng DNA/mg tissue compared to 111.78±5.18 ng/mg in the control NP, suggesting that native cells and antigens were successfully removed (FIG. 2A). The Blyscan assay demonstrated retention of 74.0% of sGAGs in the decellularized NP compared to the control NP samples, with control NP samples having sGAG content of 50.27±5.40 μg/mg and the decellularized NP samples having 37.19±5.27 μg/mg (FIG. 2B). The hydroxyproline assay results suggested that collagen took up a greater proportion of the total mass of the decellularized NP compared to control NP (146.9±54.7 control NP vs 227.3±49.1 μg/mg decellularized NP), however the decellularized and control NP were not significantly different from each other (FIG. 2C). Enrichment of collagen in decellularized NP compared to control NP was due to removal of cells and other antigens thereby reducing the overall mass, while maintaining the amount of collagen in the tissue. The ratio of sGAG:hydroxyproline was calculated to be 10.16:1 in control tissue and 4.85:1 in decellularized NP.

With regard to the cytotoxicity of remaining chemicals after decellularization, control NP cells exhibited a reduction of alamarBlue of 92.8±3.36%, whereas NP cells treated with eluted media had a reduction of alamarBlue of 80.28±9.30%, which is a significant difference. When normalized to control, NP cells treated with eluted media had a metabolic activity of 86.5±10.0% (FIG. 2D). The eluted NP cells have a 14% reduction in metabolic activity, which is below the recommended value by ISO 10993:5 for cytotoxicity. Cytotoxicity of the final re-gelled NP on DRG cultures were further assessed below during neuroinhibition studies.

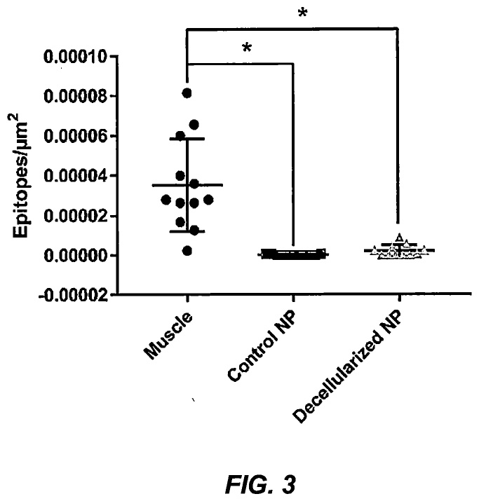

Quantification of α-gal epitopes revealed very few α- were present in either the control or decellularized NP samples. Analysis of α-gal epitopes revealed there was a significant reduction in the number of α-gal epitopes in the control NP samples and decellularized NP samples compared to the muscle tissue, but no difference between control and decellularized NP (FIG. 3).

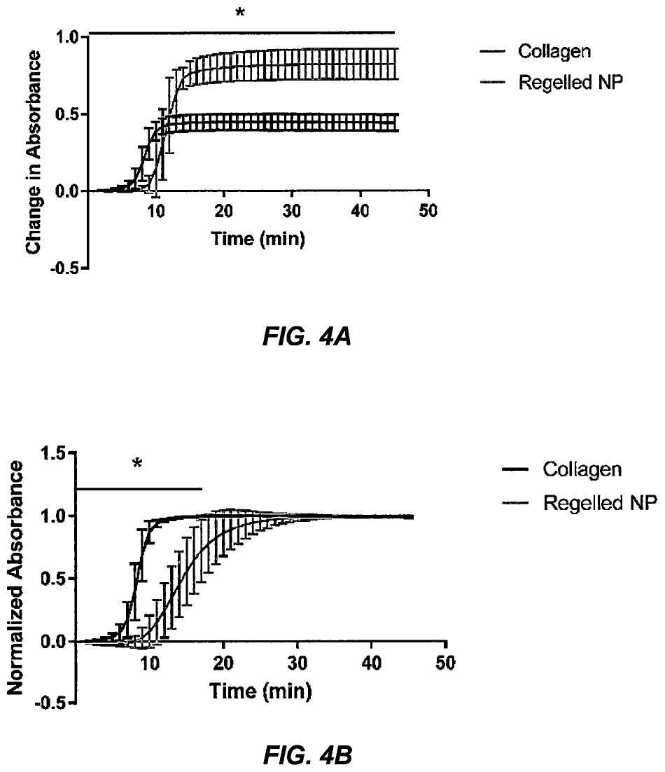

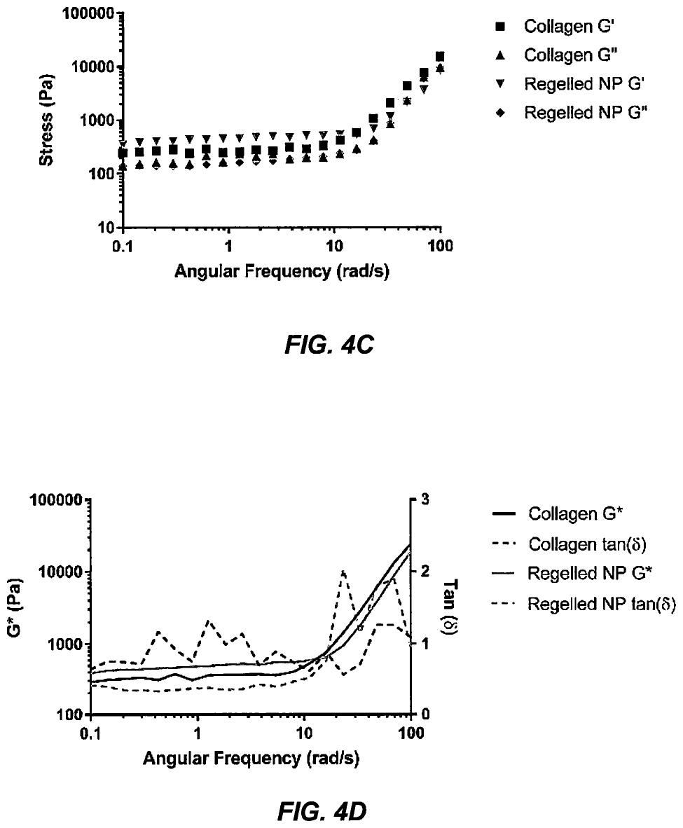

Creation and Characterization of Re-gelled NP—The normalized gel absorbance was used to determine the start and end of the gelation process. Collagen gels started and finished forming before the re-gelled NP, starting at 3 minutes, and reaching 95% of its maximum value after 12 minutes. The re-gelled NP started forming later at 8 minutes and reached 95% of its maximum value after 17 minutes (FIG. 4A and FIG. 4B). Rheology comparing the storage (G′) and loss (G″) moduli between collagen and re-gelled NP, suggests that re-gelled NP is weaker than collagen. There were no significant differences between the G″ of either group but the G′ of collagen was significantly different than that of re-gelled NP at 69.5 and 100 rad/s (FIG. 4C). Results from rheological analysis of the re-gelled NP and control collagen displayed a stable dynamic shear modulus (400-530 Pa and 300-400 Pa respectively) and tan δ (0.4-0.7 for both re-gelled NP and collagen) from 0.1 to 20 rad/s followed by an increase up to 100 rad/s where all samples had a dynamic shear modulus of ˜11,000-12,000 Pa and a tan δ of 0.72-1.4 for the re-gelled NP and 17,500 Pa and a tan δ of 0.63 for the collagen (FIG. 4D). In comparison, human NP ranges in dynamic shear modulus from 7.4-19.8 kPa and tan δ of 0.424-0.577.

SEM imaging revealed that collagen gels tended to have smaller, less organized fibers, compared to the other gels whereas control NP tissue had thicker, more dense collagen fibers, as well as cell debris covering much of the fiber surface. Decellularized NP had a looser fiber network, and the fibers were not as entangled compared to the other gels. The final re-gelled NP had thick fibers, similar to control NP, although fibers were less dense, more similar to decellularized NP. The diameters of the fibers were quantified for each group and were found to have no significant differences between any group, although re-gelled NP tended to have slightly thicker fibers compared to the other groups (FIG. 5).

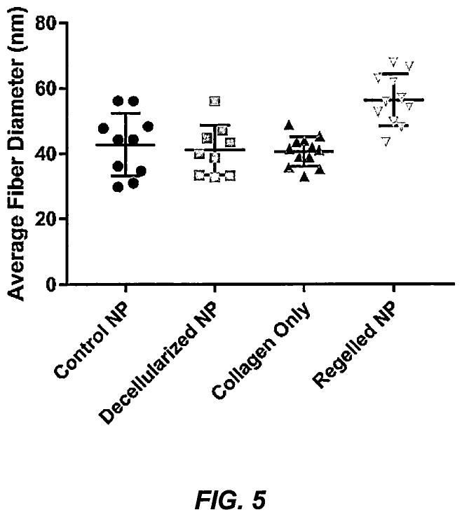

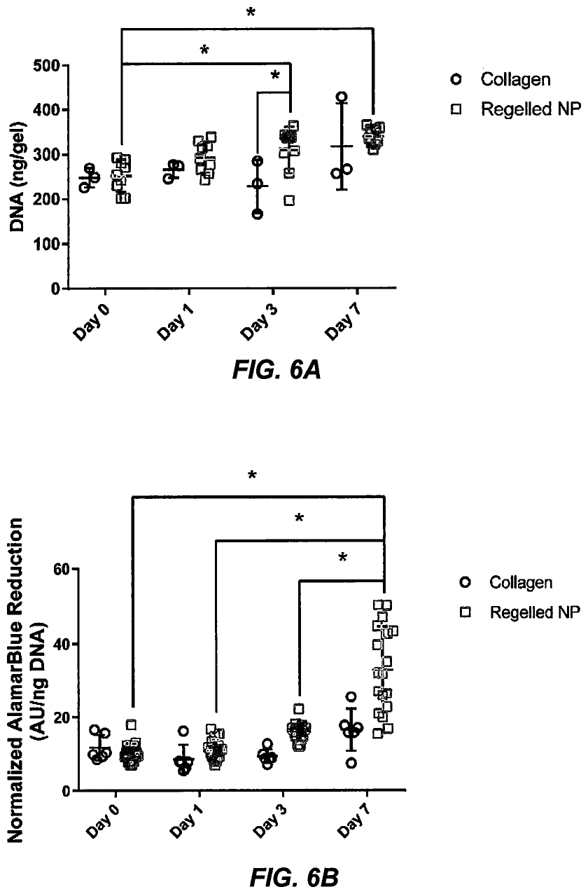

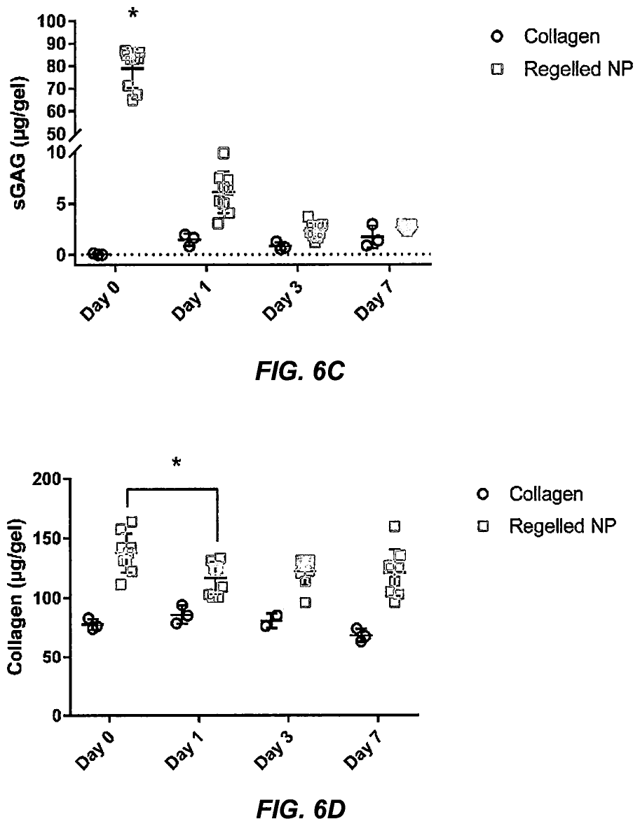

Re-gelled NP exhibited significant increases in DNA on days 3 and 7 compared to day 0, while the collagen group was significantly lower than the re-gelled NP group on day 3 (FIG. 6A). Increasing DNA indicated the gels do not inhibit cell proliferation. Normalized alamarBlue results showed that metabolic activity of human NP cells on day 7 cultured in re-gelled NP was significantly higher than every other day of that same gel (FIG. 6B). Normalized metabolic activity for day 7 re-gelled NP was also significantly greater than the day 7 collagen (FIG. 6B). The results of sGAG quantification reveal that re-gelled NP samples had a large amount of sGAG at day 0, which reduced over the first 3 days and subsequently started to slowly increase over time, although not significantly (FIG. 6C). The control collagen gels have low sGAG that gradually increased over time, although not significantly. The hydroxyproline assay demonstrated a significant but minor decrease between days 0 and 1 in the re-gelled NP group, but there were no other differences within either group over the time course (FIG. 6D). The collagen group was significantly lower than the re-gelled NP group at each time point, but also did not exhibit any differences over the time course. These data demonstrate a constant collagen content over the 7-day time course for both samples. The sGAG:hydroxyproline ratio on day 0 was 4.25:1 and reduced to 0.162:1 by day 7.

The metabolic activity of the cells in the re-gelled NP increased over time compared to the collagen group, with a significant difference by day 7, suggesting that the cells in the re-gelled NP are more active compared to the collagen group. This may be due to the cells recognizing the decellularized tissue and acting on cues that the tissue gives the cells, whereas the collagen gel would not have these same cues due to the lack of decellularized tissue. Similar phenomena have been demonstrated with cells on other decellularized tissues, with increases in metabolism by these cells when cultured on the decellularized tissue. Increases in anabolic gene expression have also been demonstrated with stem cells cultured on decellularized NP tissue compared to controls, which would suggest an increase in metabolism as well.

The DNA assay showed a small but significant increase over time in the concentration of DNA in the re-gelled NP compared to collagen controls that suggested that there was minimal cell death and the cells continued to proliferate over the course of the study. The significant increase in DNA in the re-gelled NP but not the collagen over 7 days suggested that some components in the decellularized NP was giving cues to the NP cells to proliferate, as the presence of decellularized tissue was the only difference between the two groups. Interestingly, there was a significant decrease in sGAG observed from day 0 to 1. This may be due to diffusion of the sGAGs into the media due to the large surface area exposed to media in the 30 μL gels. The re-gelled NPs cultured with sensory neurons studies still exhibited robust neuroinhibitory properties, suggesting the resultant sGAG loss is either less in these larger gels, or the loss is not sufficient to reduce neuroinhibitory properties.

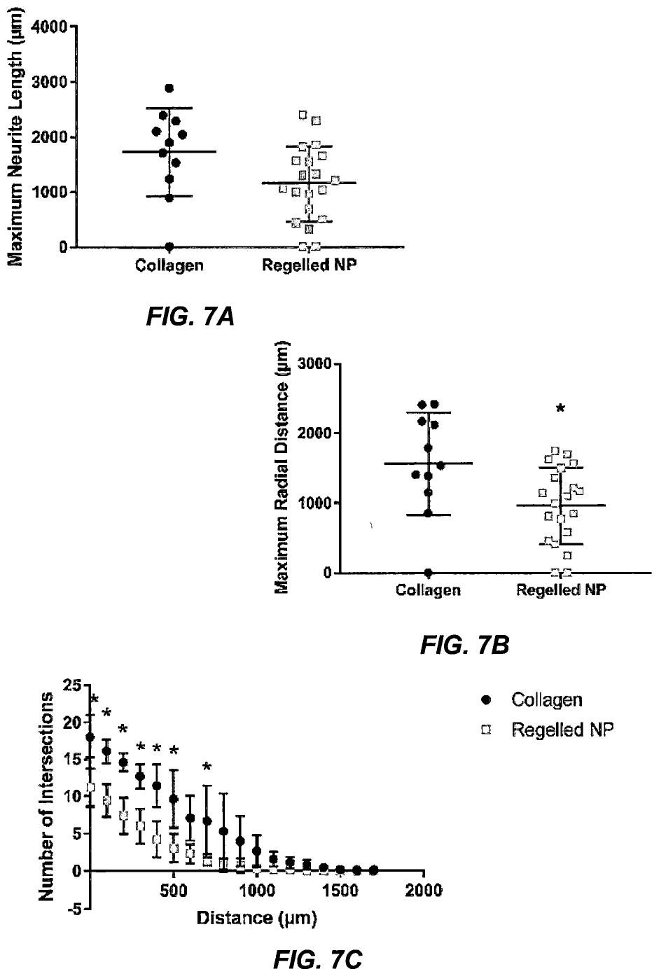

Evaluation of Neuroinhibitory Properties of Re-gelled NP—DRG neuroinhibition results demonstrated a significant reduction in the number and distance of neurites growing into re-gelled NP compared to collagen controls. Maximum neurite length in the inner gels was lower in re-gelled NP compared to collagen controls (1201.1±258.3 μm vs. 1763.9±407.5 μm) although this difference was not significant (FIG. 7A). Analysis revealed there was a significant reduction in the maximum radial distance, or the linear distance from the neurite crossing the boundary to the tip, of neurites in re-gelled NP samples compared to collagen controls (975.9±41.64 μm vs. 1601.3±381.1 μm) (FIG. 7B). The Sholl analysis found that there were significantly fewer neurons in the re-gelled NP compared to collagen at all distances up to 700 μm away from the gel boundary, except at 600 μm (FIG. 7C). The second Sholl analysis demonstrated no significant differences in the number of neurites between collagen or re-gelled NP at any distance on the opposite side of the inner gel. The average distance of the DRGs from the boundary of each group were also calculated as 278.4±118.3 m for collagen and 244±140.9 for re-gelled NP m. These distances were not significantly different from each other. These data indicate that any residuals left in the re-gelled NP are not cytotoxic to neurons and their support cells in culture. Taken together, these data demonstrate the re-gelled NP has robust neuroinhibitory properties compared to collagen controls. These data further demonstrated that the re-gelled NP was significantly more neuroinhibitory than collagen control gels.

The rheology data indicated that re-gelled NP and collagen have similar mechanical properties, therefore, mechanics is likely not a major contributor to the neuroinhibition seen in the re-gelled NP compared to collagen. The next contributing factor to neuroinhibition is the porosity of the material. A porosity of ˜80% was shown to promote the greatest nerve density and length with these values decreasing at 70 and 90% in a rat hemisection lesion. SEM analysis revealed no significant difference in fiber diameter. Although porosity cannot be calculated from SEM images, macroscopic evaluation of fibers and void space suggested similar fiber density and thus likely similar porosity between all groups. Another factor contributing to neuroinhibition was the presence of neuroinhibitory molecules in the material. Some common neuroinhibitory molecules include aggrecan, a proteoglycan present in the NP, chondroitin-6-sulfate, a major sGAG found on proteoglycans in the NP, and semaphorin3A, an axon guidance molecule in the nervous system. While aggrecan and chondroitin-6-sulfate have been found in large quantities in the NP, semaphorin3A is demonstrated more in the outer annulus fibrosus with ˜80% of cells expressing it, with a decreasing concentration into the NP, with only ˜5% of cells expressing it. Given that our re-gelled NP likely includes higher amounts of these neuroinhibitory molecules than type I collagen, the presence of sGAGs and semaphorin 3A likely contributed to the neuroinhibitory properties of the re-gelled NP. The Example demonstrates that the methods of the disclosure successfully optimized a high throughput decellularization process for whole porcine NP, created a thermally gelling hydrogel from the decellularized NP, and demonstrated the re-gelled NP gels maintain cell viability and prevents nerve growth to be used as a neuroinhibitory supplement for the NP.

The re-gelled NP had similar levels of sGAG to that of degenerate NP, however, it appeared that the re-gelled NP exhibited neuroinhibitory properties, while degenerate NP did not. The sGAG:hydroxyproline ratio was 10.16:1 in control tissue, 4.85:1 in decellularized NP, and 4.25:1 in the regelled NP. These results suggested that the re-gelled NP was similar in the ratio of sGAG to collagen to decellularized NP tissue. Because the re-gelled NP was meant as a neuroinhibitory supplement, the sGAG:hydroxyproline ratio did not need to reach back to natural tissue. Further, SEM images illustrated collagen fibers of similar orientation and thickness at each stage of the decellularization process, suggesting that the process of the disclosure retains the microstructure of a native NP.

### Example 2

### Injectable Decellularized Nucleus Pulposus Tissue Restoring Disc Volume and Alleviating Axial Hypersensitivity

A further embodiment of a hydrogel-forming composition containing dNP was analyzed for restoring disc volume and alleviating axial hypersensitivity.

### Methods

Whole Disc Decellularization—Whole disc decellularization was accomplished using a previously established protocol. Cervical spines from commercial line Landrace/Yorkshire/Duroc young female pigs (˜200 days of age) were aseptically collected and frozen (−80° C.) following humane slaughter. The spines were then cleaned aseptically and the NPs of the C2-C7 intervertebral discs were surgically removed. The NPs were decellularized using a series of detergent and buffer washes. The decellularized NPs were lyophilized (FreeZone 4.5 L Freeze Dryer (7750020, Labconco)) and stored at −80° C. until use.

Digestion and preparation of decellularized NPgel—The creation of the decellularized NP gel followed a previously described method with a few exceptions. First, lyophilized decellularized NP was comminuted via cryogenic pulverization using a steel mortar and pestle and liquid nitrogen. 20 mg/mL of comminuted dNP was digested using 1 mg/mL of pepsin (P6887, Sigma-Aldrich) in 0.05 N HCl for 44 hours at 300 rpm using a magnetic stir bar. Following digestion, all steps for making the dNP hydrogel were performed on ice, until incubation at 37° C. 10×PBS was added first to the preparation to ensure a final concentration of 1×PBS after following component additions. Next, volumes to ensure 6 mg/mL dNP digest and 6 mg/mL collagen I (Ibidi, 50201) were added to the 10×PBS. The digest was then neutralized with 5 M NaOH to pH ˜7.4. Finally, genipin (TCI Chemicals, G0458-25MG) at a stock concentration of 400 mg/mL in DMSO was added to the neutralized dNP solution to yield concentrations ranging from 0 mM to 20 mM. The final formulation used in vivo consisted of: 1×PBS, 6 mg/mL collagen, 6 mg/mL dNP and 2.5 mM genipin. Throughout the Examples, this final formulation is referred to as dNP+(i.e., combination of dNP, collagen, and genipin).

Gelation kinetics: The rate at which the dNP gels achieved complete fibrillogenesis and were cross-linked by genipin was assessed using an absorbance assay. Collagen fibrillogenesis was measured using absorbance at 405 nm as previously described. Genipin cross-linking was measured using absorbance at 610 nm. This wavelength was chosen because genipin produces genipin blue, which has high absorbance at 610 nm, as part of the cross-linking process. To measure these two absorbances longitudinally, 50 μL of dNP gel with genipin ranging from 0-20 mM was prepared and pipetted in duplicate into a 96 well plate. The plate was then placed into a microplate reader, pre-heated to 37° C. and the absorbance was read once every two minutes for 12 hours at 405 and 610 nm. A total of n=3 different preparations were evaluated.

Gel rheological characterization: Rheological analysis of the dNP gels was performed to evaluate how genipin crosslinking affected mechanical properties. Rheology testing was performed on gels using an Anton Paar MCR 302 with sand blasted plates. Briefly, dNP gels were formed by pipetting 100 uL of the gel solutions into 8-mm diameter silicone molds (666305, Grace Bio-Labs). The gels were sandwiched between glass slides within the molds to prevent desiccation and incubated at 37° C. for 24 hours to allow for complete fibrillogenesis and cross-linking. After gelation, the gels were soaked in 1×PBS for 30 minutes to reach a replicable osmotic equilibrium. An amplitude sweep was conducted to determine the storage and loss modulus across strains analogous to human movement. This characterization entailed measuring the storage and loss moduli across a strain range from 0.1-1.66% at an angular frequency of 5 Hz. The average storage and loss modulus from 0.137-0.477% strain was computed as the storage and loss modulus for each gel. Gels were fabricated and tested in duplicate, and this process was repeated three times (n=3).

Motion segment rheological characterization: Rheological analysis of rat motion segments was performed to evaluate how treatment with dNP+ affected disc mechanics after injury in an ex vivo setting and 8-weeks after administration in the animal model. Rheological assessment was performed on motion segments using an Anton Paar MCR 302 rheometer with sand blasted plates. Briefly, motion segments were excised from cadavers and potted in custom 3D printed pots using Loctite 401. For the ex vivo work, L4-L5, L5-L6, and L6-S1 motion segments were used. After pot fixation, motion segments were allowed to equilibrate overnight in 1×PBS at 4° C. To analyze, motion segments were brought to room temperature and attached to the rheometer base plate and probe using labeling tape. An amplitude sweep was then conducted to determine the storage and loss modulus across strains analogous to human movement. This characterization entailed measuring the storage and loss moduli across a strain range from 0.1-1.66% at an angular frequency of 5 Hz. The average storage and loss modulus from 0.137-0.477% strain was computed as the storage and loss modulus for each motion segment. For the ex vivo work, PBS and dNP+ motion segments were then injured using the six-scrape injury method described in the injury surgery methods. These motion segments were equilibrated again overnight and mechanically assessed the following day using an amplitude sweep with the previously described parameters. Once the post-injury values were collected, the PBS and dNP+ motion segments were injected with 1×PBS or dNP+ respectively. All motion segments were placed in a humidified incubator for 8 hours at 37° C. followed by overnight equilibration in 1×PBS at 4° C. All motion segment rheology was assessed a final time and all values were normalized against baseline. Non-injured motion segments were included in the ex vivo work as additional controls.

Cytotoxic Assessment of dNP+: To evaluate the cytotoxicity of dNP+ in vitro, human NP cells were cultured on top of dNP+ gels. In brief, P3 human NP cells (4800, ScienCell) were cultured in a T75 flask (CLS430641U, Sigma Aldrich) coated with 15 μg of Poly-L-Lysine (0413, ScienCell) using Complete Nucleus Pulposus Cell Media (4801, ScienCell) till confluent to prepare NP cells for treatment plating. 48-well plates were prepared for treatment culture by coating with either nothing (PS) or with 10 μL of 6 mg/mL collagen gel, 6 mg/mL collagen+6 mg/mL dNP gel, or 6 mg/mL collagen+6 mg/mL dNP+2.5 mM (dNP+) genipin gel. The coatings were gelled for 8 hours at 37° C. in a humidified incubator and then equilibrated for 12 hours in 1×PBS. NP cells were plated on top of the treatments at a density of 7,500 cells/cm² and cultured for 48 hours in phenol free Complete Nucleus Pulposus Cell Media (4801-prf, ScienCell). After the 48 hours expired, each well was washed three times with 1×PBS and incubated with 200 μL 2 μM Calcein AM (ThermoFisher C3100MP) and 4 μM Eth-1 (ThermoFisher E1169) in 1×PBS for 30 minutes. A kill well was included for each donor as a positive control for Eth-1 staining. The cells were then washed three times with 1×PBS and imaged on a Cytation 5 (BioTek) at 10× using the following channels and settings: GFP with LED power at 3, integration at 21 ms and gain at 0, RFP with LED power at 3, integration at 87 ms, and gain at 0, and BF with LED power at 7, integration at 100 ms, and gain at 0.5. Four images were taken from each well, deconvoluted, and analyzed using Gen 5 software to evaluate particle number and size in each channel. Viability was calculated using the following calculation: (#GFP objects/(#GFP objects+#RFP objects)×100.

Animals: 36 female, 15-week-old, Sprague Dawley rats were purchased from Envigo and housed with a 12-hour light/dark cycle and ad libitum access to food and water. On the day of surgery, the animals were split into three groups of equal size (n=12): sham, PBS, and dNP+. After surgery, all animals were weighed and assessed on a weekly basis for overall health. Sample sizes were chosen to ensure sufficient power to detect a 30% decrease in grip strength in injured animals compared to sham animals assuming a standard deviation that was 26% of the mean.

Injury Surgery: On the day of injury surgery, rats were anesthetized, and the lumbar spine was approached ventrally by dissecting through the abdominal cavity and retroperitoneum. The iliac crest was used as a landmark to reliably dissect down to the L5-L6 disc. For sham animals, the L5-L6 was visualized only, and the surgical site was closed in the same manner as injured animals outlined below. For PBS and dNP+ animals, the L5-L6 lumbar disc was punctured bilaterally with a strong point dissecting needle (Roboz, RS-6066) with an O.D. of 0.5 mm set to a length of 2.75 mm. The exact needle length was predetermined based on μCT data to ensure that the needle length did not exceed the diameter of the smallest L5-L6 disc in all animals.

Treatment Surgery: On the day of surgery, rats were anesthetized, and the L5-L6 disc was approached using the same method as the injury surgery. For sham animals, the L5-L6 was visualized only, and the surgical site was closed in the same manner as treated animals. For PBS and dNP+ animals, the L5-L6 lumbar disc was injected through the midline using a 30-gauge needle (BD, 305106) with a rubber stopper (WidgetCo, 7-R0000000-EPDM-RS) fixed at 2 mm attached to a 10 μL microsyringe (Hamilton, 80001). PBS animals were injected with 2.5 μL of 1×PBS and dNP+ animals were injected with 2.5 μL dNP+. The needle length was predetermined based on μCT data to increase the probability that the therapeutic was delivered to the NP space. The injection site was sealed with Vetbond (3M, 1469c).

Disc volume: The L5-L6 disc volume was quantified using the Quantum GX2 μCT Imaging System and Analyze 13.0 (Analyze Direct). The rat lumbar spine was radiographed by placing anesthetized animals in the supine position and scanning for 2-minutes with 90 kV power, 88 μA tube current, 72 mm FOV, 144 μm voxel size, and a Cu 0.06+Al 0.05 X-ray filter. VOX files were then removed from the μCT computer and analyzed in Analyze 13.0. To begin processing, raw VOX files from the CT scans were filtered using a high pass threshold of 700 Hounsfield units to remove all non-bony signal. After the scans were reduced to only bony tissue using the software filter, the intervertebral disc space was colored in every coronal plane where the adjacent vertebral bodies were present using a manual draw tool. The slices of colored intervertebral disc space between the L5 and L6 vertebral bodies were then concatenated, smoothed using a built-in function and saved as an object map. The volume of the object map of these concatenated colored planes was quantified using a built-in software analysis. This quantification was based on a previously established method developed in our lab.

Grip strength axial hypersensitivity: Hypersensitivity to axial strain was quantified using a grip strength apparatus (Columbus Instruments, 1027SR). All animals were allowed to acclimate to the testing room for 15 minutes prior to testing. Animals were picked up by grasping the hind quarter and then allowed to grip a metal wire mesh attached to the grip strength force sensor. The experimenter's grip was then transitioned to the base of the tail and the animal was gently pulled backward until it released the metal wire mesh. This process was repeated three times and the average max force (N) was used as the grip strength. All withdraw grip strength thresholds were log transformed to achieve normality and then normalized to baseline to reduce variability.

Pressure algometry deep tissue hypersensitivity: Hypersensitivity around the L5-L6 motion segment was measured using an electronic von Frey aesthesiometer (IITC, 2391) with a blunt tip. All animals were allowed to acclimate to the testing room for 15 minutes prior to testing. Each animal was sequentially hooded inside a clean cotton t-shirt such that the entire animal was covered. The animal was then loosely constrained by one experimenter while another experimenter applied the blunt probe to the dorsal L5-L6 skin and slowly increased the pressure until the animal exhibited a nocifensive response. The L5-L6 skin area was ascertained by palpating along the caudal spinal curvature to locate the area of skin directly superficial to the spinous processes just caudal of the iliac crest. Positive responses included rolling, rapid movement, and vocalization. Two measurements were collected for each animal and the average was used as the deep tissue pressure threshold. All animal thresholds were normalized to baselines to reduce variability.

Open field test: Spontaneous pain-like behavior was evaluated using the open field test with custom built acrylic 2′×2′×2′ black, opaque arenas. All animals were allowed to acclimate to the testing room for 15 minutes prior to testing. Animals were individually placed in arenas illuminated by overhead red lighting and allowed to explore for 30 minutes while recorded by an overhead low-light camera (ALPCAM). The middle 20 minutes of each video was analyzed using Ethovision (Noldus) for total distance traveled, time spent rearing unsupported, time spent rearing supported, time spent grooming, max velocity, average turn angle, and max turn angle. All data were normalized to baselines to reduce variability.

### Results

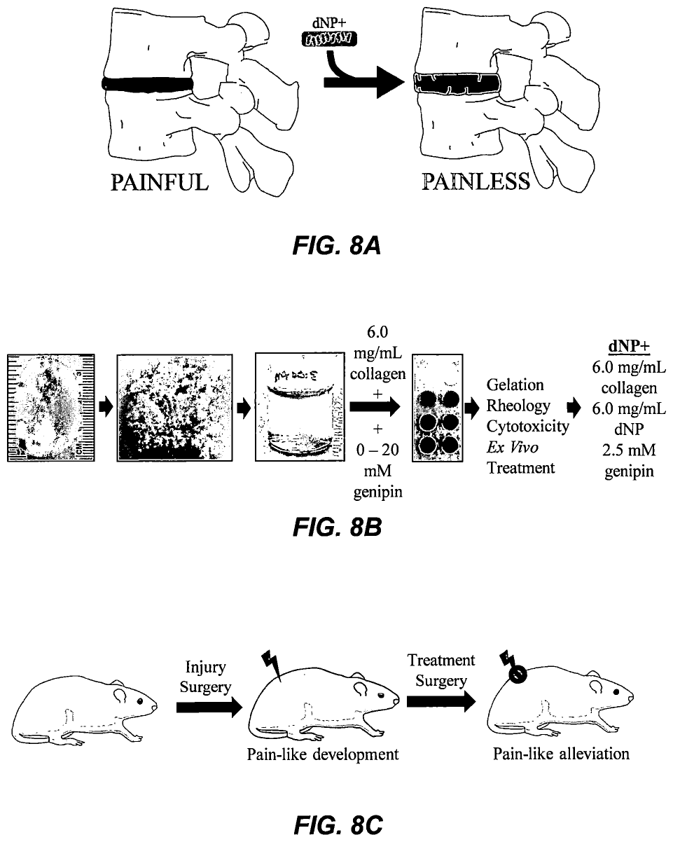

Study Overview—The central proposition of this work was that a painful degenerated disc could be transitioned to a painless state using a hydrogel fabricated from decellularized tissue (FIG. 8A). The success of this proposition hinged upon delivery and maintenance of the hydrogel within the degenerated disc. This criterion motivated the inclusion of a tissue crosslinker, genipin, to form inter-collagenous covalent bonds between the hydrogel and the degenerated disc tissue. As shown in FIG. 8B, a process of engineering a genipin crosslinked hydrogel derived from decellularized NP tissue and collagen type I was completed. To accomplish this, cervical NPs were dissected, decellularized, comminuted, digested, neutralized, and supplemented with 6 mg/mL collagen to form a dNP gel. Additionally, dNP gels were supplemented with 0-20 mM genipin to measure the variable effects of genipin crosslinking on select outcomes. These outcomes consisted of gelation kinetics, rheological properties, cytotoxicity, and ex vivo capacity to augment disc mechanics. Work done in this first arm suggested a 6.0 mg/mL dNP+6.0 mg/mL collagen I+2.5 mM genipin hydrogel, termed dNP+, was the best candidate for in vivo evaluation. To test the therapeutic in vivo, disc degeneration was induced in female Sprague Dawley rats and progressed for 8-weeks (FIG. 8C). At 9-weeks, injured animals were treated with dNP+ or 1×PBS, and pain-like alleviation was measured over the following 6 weeks.

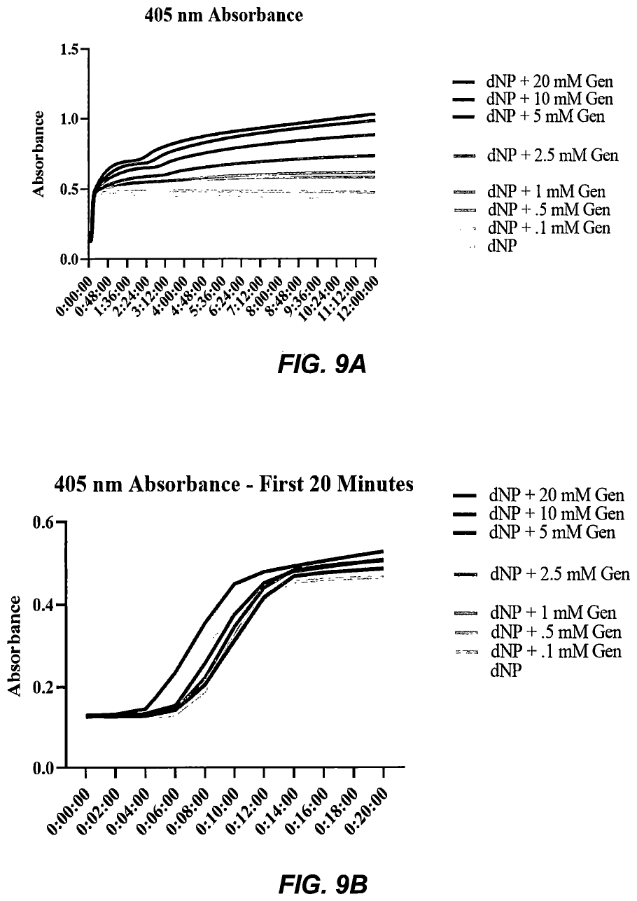

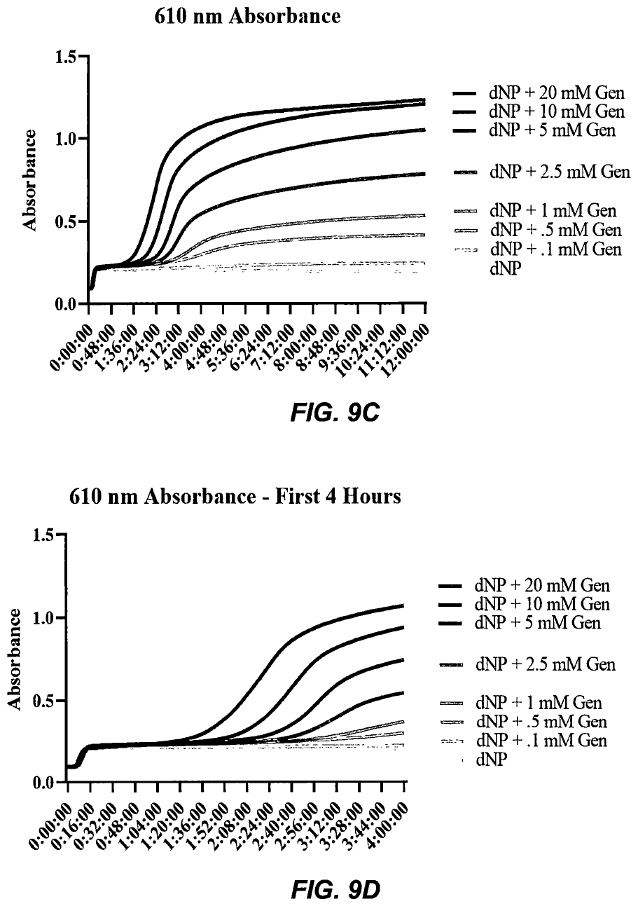

dNP hydrogels spontaneously gel and crosslink—Considering a core criterion of this therapeutic was to be spontaneously fibrillogenic after injection into a ˜37° C. tissue, gelation kinetics were first to be determined. All dNP gels completed collagen fibrillogenesis in under 15 minutes (FIGS. 9A and 9B). The rapid gelation indicated these gels would be suitable for an injection procedure because fibrillogenesis would complete in the time required for wound closure and awakening. Crosslinking mediated by genipin took considerably longer than collagen fibrillogenesis with most formulations hitting 75% max 610 nm absorbance by four hours (FIGS. 9C and 9D). Of note, the cross-linking process appeared relatively stochastic with higher genipin concentrations beginning crosslink formation earlier than lower concentrations. Further, the asymptotic limit of absorbance seen at 610 nm for the 10 mM and 20 mM genipin dNP hydrogels could be due to limitations of the plate reader, true nature of the gel absorbance, or both. All gels were homogenous in appearance and did not desiccate during the 12-hour collection window. These data showed genipin hydrogels completed fibrillogenesis in under 15 minutes and that the majority of genipin crosslinking activity was completed in 4 hours in all formulations.

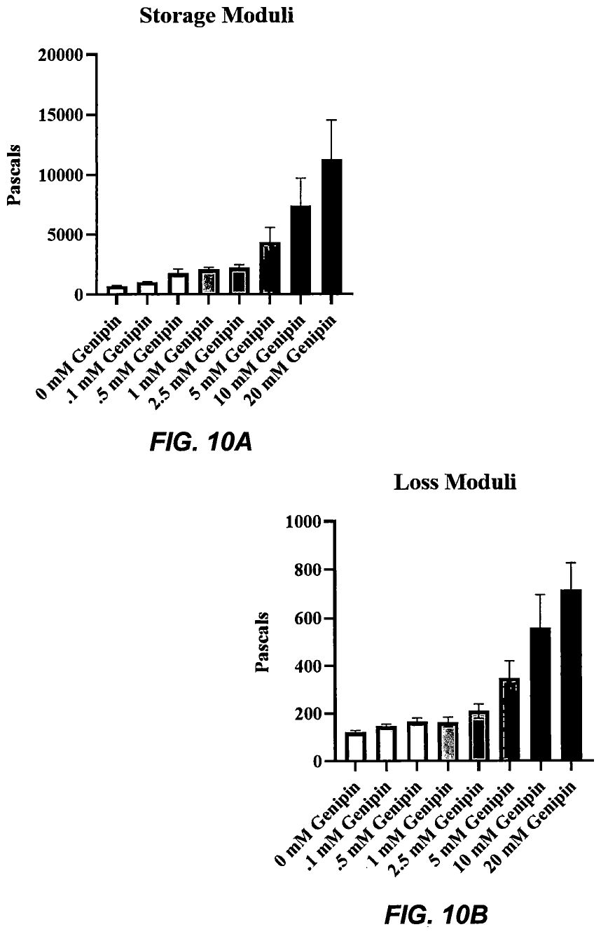

Genipin increases dNP rheological properties—Each dNP formulation was measured using a rheometer for storage and loss moduli. The average storage and loss modulus for 0 mM genipin gels was 709.81±44.0 Pa and 118.19±8.68 Pa respectively (FIGS. 10A and 10B). The addition of genipin markedly increased storage and loss moduli with 20 mM genipin increasing the storage modulus more than 10-fold (11271±3248.0 Pa) compared to 0 mM gels. The average storage and loss modulus for the 2.5 mM genipin dNP gel were 2244.6±208.60 Pa and 210±30.976 Pa respectively. Interestingly, the dNP hydrogels suffered increasing variability at genipin concentration of 5 mM and above. It is unclear what drove this increase in variability as duplicate variability and inter experimental variability were both present at these higher concentrations. Unlike the gelation kinetic data, the rheological properties did not reach an asymptotic limit indicating that cross-linking sites were not exhausted at high genipin concentrations. Overall, these data confirmed genipin increased dNP mechanical properties and that 2.5 mM genipin was the highest concentration usable to maintain replicable fabrication outcomes.

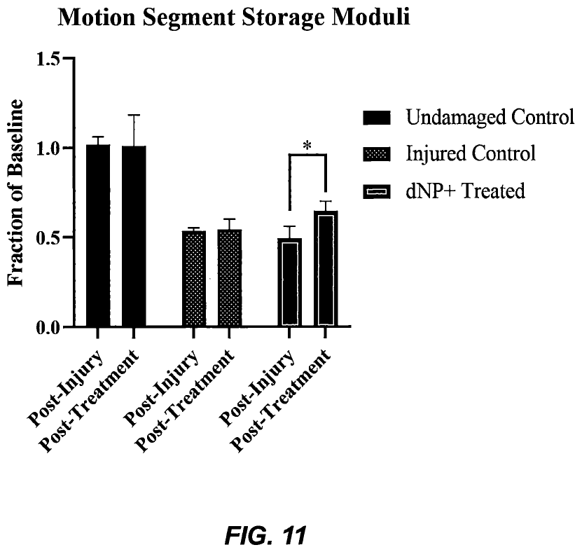

dNP+ increases injured motion segment rheological properties—Two additional necessities for dNP+ were injectability and capacity to alter disc mechanics. These two aspects were tested ex vivo using motion segments excised from female Sprague Dawley rat cadavers. The injection process was specifically designed to include sterilizable, single-use needles and rubber stoppers such that the injection depth could be tuned, and sterility could be maintained for in vivo injections in the animal model. 30-gauge needles with rubber stoppers fixed to the needle with cyanoacrylate paired with 10 μL microinjection syringes proved most effective in providing a consistent, easy, and straightforward injection process. To determine how well dNP+ restored disc mechanics, the rheological properties of potted motion segments were assessed at baseline, after 6-scrape injury, and after treatment (FIG. 11). Undamaged controls were included at all time points. All data was normalized to pre-injury baselines so that motion segments from L4 to S1 could be pooled without dramatically increasing the variability. Injury decreased the storage modulus of motion segments by around 50% compared to baseline. Treatment with dNP+ significantly restored the modulus by 15% compared to post-injury (p<0.05). These data confirmed dNP+ alone could increase disc mechanical properties after injury.

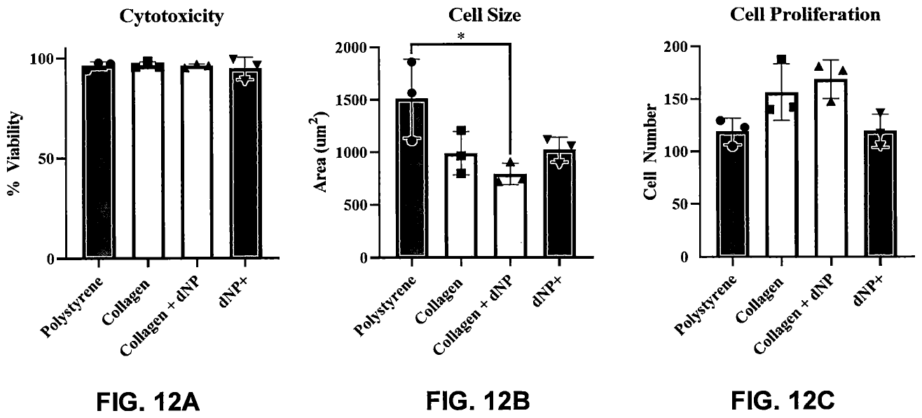

dNP+ is cytocompatible—To understand if the dNP+ hydrogel was cytotoxic, human NP cells were cultured on top of dNP+ for 48 hours and viability was determined using Calcein AM and Eth-1. Polystyrene, collagen, and collagen+dNP substrates were included as controls. All treatments exhibited minimal cell death due to the absence of Eth-1 fluorescence. When quantified, the viability for polystyrene, collagen, collagen+dNP, and dNP+ treatments was 96.37±1.91%, 96.54±1.66%, 96.15±0.91%, and 94.75±5.61% respectively (FIG. 12A). Cell size was also quantified to assess how each substrate impacted cellular morphology (FIG. 12B). Unsurprisingly, the collagen+dNP control which represented a less stiff substrate compared to polystyrene caused the cells to adopt significantly smaller morphology compared to polystyrene. However, this effect was partially reversed with the addition of 2.5 mM genipin in the dNP+ treatment. Rheological data indicated 2.5 mM genipin made the hydrogel stiffer which likely caused the cells to adopt a more elongated morphology. Finally, total cell count was measured to understand if dNP+ had a negative effect on cellular proliferation (FIG. 12C). Analysis of the cell counts indicated dNP+ did not significantly decrease cellular proliferation compared to collagen+dNP although the difference narrowly missed significance (p<0.052). These results demonstrated that dNP+ is not cytotoxic to human NP cells but the trend toward decreased cellular proliferation suggested concentrations of genipin above 2.5 mM might be harmful to cell growth and perhaps viability.

Animal Study Overview—A rat model of disc-associated pain, in particular, one that develops robust axial hypersensitivity after disc injury, was employed to evaluate the efficacy of dNP+ as a rescue treatment for disc-associated low back pain. This arm entailed three weeks of acclimation, injury surgery at week 0, eight weeks of data collection, treatment surgery at week 9, six weeks of data collection, and post-processing at week 15+. For the study, 36, female Sprague Dawley rats were split into three groups of equal size: sham—12, PBS—12, dNP+−12. One sham animal was euthanized prematurely due to surgical complications and one dNP+ animal was excluded due to incorrect treatment injection. Animal pain-like development was assessed using two evoked pain-like behavior assays, grip strength and pressure algometry, and one spontaneous pain-like behavior assay, the open field test. Disc degeneration was induced at week 0 in PBS and dNP+ animals and involved mechanical injury of the L5-L6 IVD using a ventral approach. Sham discs were visualized only. The treatment surgery followed the same steps as the injury surgery to visualize the disc. PBS and dNP+ animal discs were injected with 2.5 uL of 1×PBS or dNP+ respectively. Sham discs were visualized only.

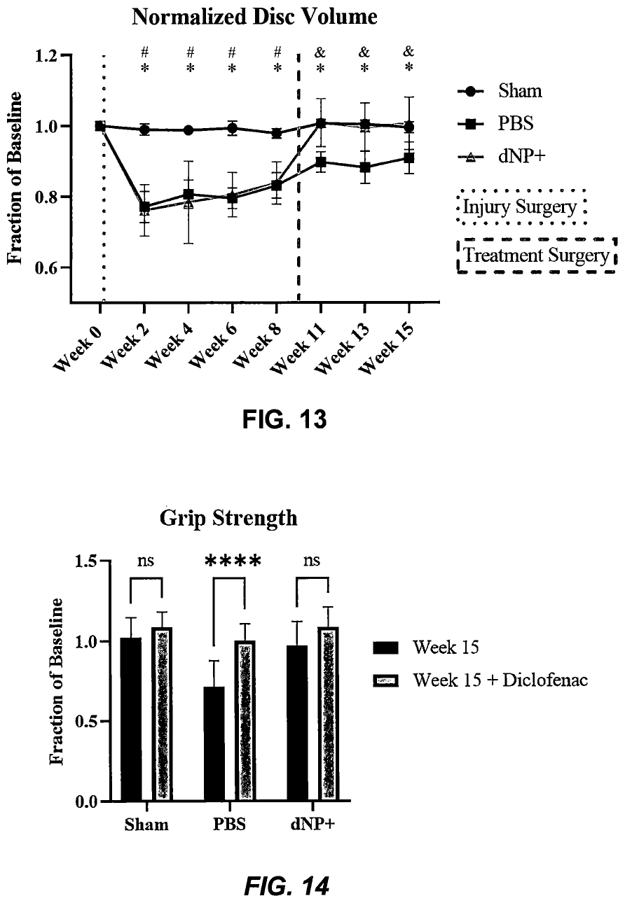

dNP+ restores disc volume—Intervertebral disc volume was quantified at eight time points as a real time measurement of disc degeneration (FIG. 13). After injury, the AF was unable to function as a hydrostatic barrier between the highly hydrated NP and surrounding tissue. This loss of function caused the disc to reach a new osmotic equilibrium with the surrounding tissue resulting in a loss of disc height and volume. From weeks 2 to 8, both PBS and dNP+ groups exhibited significantly decreased disc volume compared to sham animals. After treatment, dNP+ disc volume returned to baseline and was not significantly different from sham at any time point from week 11 till study conclusion. Conversely, IVDs injected with 1×PBS remained at a sub baseline volume and were significantly different from sham and dNP+ at all time points. These data demonstrated dNP+ treatment restored disc volume 9 weeks after degeneration was initiated.

Deep pressure hypersensitivity as measured by the pressure algometry assay failed to detect differences between sham and dNP+ groups at any time point. However, this assay was successful in detecting differences between PBS and sham animal groups at weeks 4, 8, 13, and 15. Also, animals in the PBS group displayed significantly decreased thresholds compared to dNP+ animals at week 13, 4 weeks after treatment surgery. Data from this assay suggests dNP+ may have been effective in decreasing deep pressure hypersensitivity but the failure of dNP+ animals to develop deep pressure hypersensitivity prior to treatment, limits the interpretability of this data set.

The open field test measured changes in spontaneous pain-like behavior. The only time point at which significant differences in total distance travel were observed was at week 11, 2 weeks after treatment surgery. At this point dNP+ animals traveled significantly further than both PBS and sham groups. This highly significant difference, suggested the dNP+ treatment may have alleviated nociception evoked through movement, causing the animals to roam more.

Finally, an NSAID known to effectively treat acute low back pain in humans, diclofenac, was administered intraperitoneally to all animals in week 15 to determine if disc-associated pain could be alleviated in the animal model via drugs used in humans (FIG. 14). This evaluation was crucial for providing evidence of this model's predictive validity. After diclofenac administration, PBS animal grip strengths returned to baseline and were significantly different from week 15 pre-treatment evaluation. These data confirmed pain-like hypersensitivity in this model could be alleviated via approaches used in humans and that the increase in grip strength observed after treatment with dNP+ was likely due an alleviation of nociception in the degenerated disc tissue. Altogether these pain-like measurements indicate dNP+ was effective at alleviating pain-like hypersensitivity and the effect of dNP+ was sustained for 6 weeks after treatment.

The above specification provides a description of the manufacture and use of the disclosed compositions and methods. Since many embodiments can be made without departing from the spirit and scope of the disclosure, the disclosure resides in the claims.

What is claimed is:

1. A hydrogel-forming composition comprising:
a decellularized nucleus pulposus tissue (dNP); and
a collagen,
wherein the composition is a hydrogel-forming composition.

2. The composition of claim 1, wherein the dNP is present in the composition in an amount of between about 1 mg/mL to about 15 mg/mL.

3. The composition of claim 1, wherein the collagen comprises type I collagen, and is present in the composition in an amount of from about 1 mg/mL to about 9 mg/mL.

4. The composition of claim 1, wherein the composition further comprises a crosslinking agent comprising a genipin crosslinking agent, a lysyl oxidase (LOX) crosslinking agent, or a combination thereof.

5. The composition of claim 4, wherein when the crosslinking agent comprises the genipin crosslinking agent, the genipin crosslinking agent is present in the composition in an amount of from about 0.1 mM to about 20 mM, and wherein when the crosslinking agent comprises the LOX crosslinking agent, the LOX crosslinking agent is present in the composition in an amount of from about 0.02 ng/mL to about 2 ng/mL.

6. The composition of claim 1, wherein the dNP is a digested dNP, wherein the digested dNP is formed via a treatment of the dNP with an enzyme comprising pepsin in a concentration range of between about 1 mg/mL to about 10 mg/mL.

7. The composition of claim 1, wherein the composition has a pH of between about 6 and about 8.

8. A method of treating pain or inflammation in a subject, the method comprising:
administering a hydrogel-forming composition to a subject, wherein the composition comprises a decellularized nucleus pulposus tissue (dNP) and a collagen,
wherein the administration of the composition to the subject prevents, eliminates, or reduces symptoms of pain or inflammation in the subject.

9. The method of claim 8, wherein the hydrogel-forming composition further includes a crosslinking agent comprising a genipin crosslinking agent, a lysyl oxidase (LOX) crosslinking agent, or a combination thereof.

10. The method of claim 9, wherein the crosslinking agent aids in forming inter-collagenous covalent bonds between the hydrogel-forming composition and adjacent intervertebral disc tissue of the subject.

11. The method of claim 8, wherein the subject is a human, a dog, a cat, or a horse, and wherein the hydrogel-forming composition is administered to the subject via an injection.

12. The method of claim 11, wherein the hydrogel-forming composition is administered to a joint of the subject, wherein the joint is a synovial joint, vertebral joint, temporomandibular joint, sacroiliac joint, finger or toe joint, ankle joint, elbow joint, hip joint, or a combination thereof.

13. The method of claim 8, wherein the hydrogel-forming composition is administered into an intervertebral disc of the subject.

14. The method of claim 13, wherein the hydrogel-forming composition is neuroinhibitory, and wherein the hydrogel-forming composition prevents nerve growth, increases intervertebral disc volume, increases intervertebral disc mechanics, or a combination thereof, resulting in the prevention, elimination, or reduction of symptoms of pain or inflammation in the subject.

15. The method of claim 8, wherein the pain or inflammation in the subject is a result of low-back pain, neck pain, or a combination thereof.

16. The method of claim 8, wherein the composition is administered to the subject as a single administration, or once every one to ten years.

17. A method of making a composition, the method comprising:
digesting a decellularized nucleus pulposus tissue (dNP) to form a digested dNP via a treatment of the dNP with an enzyme; and
combining the digested dNP with a collagen to form a dNP gel.

18. The method of claim 17, further comprising a step of combining a crosslinking agent comprising a genipin crosslinking agent, a lysyl oxidase (LOX) crosslinking agent, or a combination thereof to the dNP gel to form a hydrogel-forming composition.

19. The method of claim 17, wherein the dNP is present in the composition in an amount of between about 1 mg/mL to about 15 mg/mL, wherein the enzyme comprises pepsin in a concentration of between about 1 mg/mL to about 10 mg/mL, and wherein the collagen comprises type I collagen in an amount of from about 1 mg/mL to about 9 mg/mL.

20. The method of claim 17, wherein the digested dNP is further neutralized with a basic agent to result in a pH of between about 6 and about 9 prior to combining with the collagen.
